(19) United States
(12) Patent Application Publication
(10) Pub. No.: US 2018/0214560 A1
(43) Pub. Date: Aug. 2, 2018

(54) HPMA-DRUG CONJUGATES FOR THE TREATMENT OF ACUTE MYELOID LEUKEMIA

(71) Applicant: University of Utah Research Foundation, Salt Lake City, UT (US)

(72) Inventors: Paul Shami, Sandy, UT (US); Jindrich Kopecek, Salt Lake City, UT (US); Jiyuan Yang, Salt Lake City, UT (US); Rui Zhang, Salt Lake City, UT (US)

(21) Appl. No.: 15/747,413

(22) PCT Filed: Jul. 22, 2016
(86) PCT No.: PCT/US16/43732

§ 371 Date: Jan. 24, 2018

Related U.S. Application Data
(60) Provisional application No. 62/196,372, filed on Jul. 24, 2015.

(51) Int. Cl. A61K 47/58; A61P 35/02; A61K 31/7068; A61K 31/5377; A61K 47/68; C07K 16/28; A61K 39/395; A61K 31/704

(52) U.S. Cl. CPC A61K 47/58; A61P 35/02; A61K 31/7068; A61K 31/5377; A61K 47/6807; A61K 47/6803; A61K 2039/505; A61K 39/3955; A61K 47/6849; A61K 47/6883; A61K 31/704; C07K 2317/55; C07K 16/2803

(57) ABSTRACT

Disclosed are methods are of treating acute myeloid leukemia (AML) comprising administering an effective amount of a first AML therapeutic and an effective amount of a second AML therapeutic. Also disclosed are methods of treating AML comprising administering an effective amount of a first AML therapeutic conjugated to N-(2-hydroxypropyl)methacrylamide (HPMA) copolymer. Also disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence.

### CROSS REFERENCE TO RELATED APPLICATIONS

This application claims the benefit of the filing date of U.S. Provisional Application No. 62/196,372, which was filed on Jul. 24, 2015. The content of this earlier filed application is hereby incorporated by reference herein in its entirety.

### BACKGROUND

The mainstay therapy for acute myeloid leukemia (AML) is the combination of cytarabine and an anthracycline. This regimen was originally developed about 4 decades ago and remains the standard of care. In spite of significant advances in understanding AML, the majority of patients died from their disease. The median age at diagnosis is 66 years and overall survival for senior AML patients has not changed in the past 30 years, with cure rates less than 10% and median survival less than 1 year. Although about 80% of patients younger than 60 can get complete remissions, most eventually relapse and 5-year survival is only 40-50% in that age group. Therefore, the need for new treatment strategies for AML is evident.

### BRIEF SUMMARY

Disclosed are methods of treating AML comprising administering an effective amount of a first AML therapeutic and an effective amount of a second AML therapeutic. The first AML therapeutic can be cytarabine, daunorubicin, GDC-0980, arylated diazeniumdiolate, or derivatives thereof. The second AML therapeutic can be different from the first AML therapeutic, wherein the second AML therapeutic can be cytarabine, daunorubicin, GDC-0980, arylated diazeniumdiolate, or derivatives thereof. For example, at least one of the AML therapeutics can be cytarabine or GDC-0980.

Disclosed are methods of treating AML comprising administering an effective amount of a first AML therapeutic and an effective amount of a second AML therapeutic, wherein the first and second AML therapeutics provide a synergistic effect.

Disclosed are methods of treating AML comprising administering an effective amount of a first AML therapeutic and an effective amount of a second AML therapeutic, wherein the first and second AML therapeutics are formulated in a single composition.

Disclosed are methods of treating AML comprising administering an effective amount of a first AML therapeutic and an effective amount of a second AML therapeutic, wherein the first and second AML therapeutics are formulated in separate compositions.

Disclosed are methods of treating AML comprising administering an effective amount of a first AML therapeutic and an effective amount of a second AML therapeutic, wherein the first and second AML therapeutics are formulated in separate compositions, wherein the first and second AML therapeutics are administered simultaneously.

Disclosed are methods of treating AML comprising administering an effective amount of a first AML therapeutic and an effective amount of a second AML therapeutic, wherein the first and second AML therapeutics are formulated in separate compositions, wherein the first and second AML therapeutics are administered consecutively.

Also disclosed are methods of treating AML comprising administering an effective amount of a first AML therapeutic conjugated to N-(2-hydroxypropyl)methacrylamide (HPMA) copolymer.

Disclosed are methods of treating AML comprising administering an effective amount of a first AML therapeutic conjugated to HPMA copolymer, further comprising a second AML therapeutic. In some instances, the second AML therapeutic is conjugated to HPMA copolymer.

Disclosed are methods of treating AML comprising administering an effective amount of a first AML therapeutic conjugated to HPMA copolymer, further comprising a second AML therapeutic, wherein the first and second AML therapeutics provide a synergistic effect.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises cytarabine, daunorubicin, GDC-0980, arylated diazeniumdiolate, or derivatives thereof.

Disclosed are methods of treating AML comprising administering an effective amount of a first AML therapeutic conjugated to HPMA copolymer, further comprising a second AML therapeutic, wherein the second AML therapeutic conjugated to HPMA copolymer comprises cytarabine, daunorubicin, GDC-0980, arylated diazeniumdiolate, or derivatives thereof. In some instances, the second AML therapeutic conjugated to HPMA copolymer is different than the first AML therapeutic conjugated to HPMA copolymer.

Disclosed are methods of treating AML comprising administering an effective amount of a first AML therapeutic conjugated to HPMA copolymer, further comprising a second AML therapeutic, wherein at least one of the AML therapeutics conjugated to HPMA copolymer is cytarabine.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer are formulated in a single composition.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer are formulated in separate compositions. In some instances, the first and second AML therapeutics conjugated to HPMA copolymer are administered simultaneously. In some instances, the first and second AML therapeutics conjugated to HPMA copolymer are administered consecutively.

Disclosed are methods of treating AML comprising administering an effective amount of a first AML therapeutic conjugated to HPMA copolymer, further comprising a second AML therapeutic, wherein at least one of the AML therapeutics conjugated to HPMA copolymer comprise a GFLG linker.

Disclosed are methods of treating AML comprising administering an effective amount of a first AML therapeutic conjugated to HPMA copolymer, further comprising a second AML therapeutic, wherein at least one of the AML therapeutics conjugated to HPMA copolymer further comprises a targeting moiety. In some instances, the targeting moiety is an antibody or fragment thereof.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the second AML therapeutic conjugated to HPMA copolymer comprises a HPMA copolymer backbone comprising at least two HPMA copolymer segments connected by degradable peptide sequences.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the first AML therapeutic conjugated to HPMA copolymer comprises at least two AML therapeutics conjugated to the HPMA copolymer backbone.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the second AML therapeutic conjugated to HPMA copolymer comprises at least two AML therapeutics conjugated to the HPMA copolymer backbone.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the AML therapeutics conjugated to HPMA copolymer comprise a GFLG linker.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the HPMA copolymer backbone comprises a GFLG linker.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the HPMA copolymer backbone further comprises a targeting moiety. In some instances, the targeting moiety comprises an antibody or fragment thereof. In some instances, the antibody of fragment thereof comprises an Fab region. In some instances, the antibody or fragment thereof is an anti-CD33 antibody or fragment thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the in vitro cytotoxicity of the free drugs (CYT, DAU, GDC, and JSK) toward HL-60 human AML cells. The IC₅₀ values are presented as mean±standard deviation (n=3).

FIG. 2 shows the combination index (CI) of two drugs. HL-60 leukemia cells were treated with different two-drug combinations, including CYT+DAU, CYT+GDC, CYT+JSK, DAU+GDC, DAU+JSK, and GDC+JSK. A constant ratio was used in this combination setting (CYT:DAU:GDC:JSK=50:2:50:25). CI was calculated by Chou-Talalay method. Data plotted are CI values at 25, 50, 75, and 90% Fa (fraction affected). All the data are expressed as mean±standard deviation (n=3).

FIG. 3 shows the percentage of HL-60 leukemia cells in the different phases of cell cycle after single-drug treatment or two-drug combinations. HL-60 cells were treated with individual drug alone or the combination of two drugs (CYT=1 μM; DAU=0.04 μM; GDC=1 μM; JSK=0.5 μM) for 48 h. Cell cycle analysis was performed by flow cytometry after propidium iodide staining. All the data are expressed as mean±standard deviation (n=3).

FIG. 4 shows the synthetic scheme of HPMA copolymer-drug conjugates (P-CYT and P-GDC) via RAFT polymerization. The synthesis of both conjugates used 4-cyanopentanoic acid dithiobenzoate as the RAFT chain transfer agent followed by end modification with V65. The synthesis of P-CYT had V-501 as the initiator, while P-GDC used VA-044 as the initiator.

FIG. 5 shows the decrease of CYT concentration (in percentage) with time in human plasma as free drug or in HPMA copolymer-CYT conjugate.

FIGS. 6A and 6B shows the in vitro cytotoxicity (A) and combination index (B) of the HPMA copolymer-drug conjugates (P-CYT and P-GDC). HL-60 leukemia cells were incubated with individual conjugate (P-CYT, P-GDC) or both conjugates (P-CYT+P-GDC) simultaneously for 48 h. C The molar radio of CYT to GDC was set as 1:1. CI was calculated by Chou-Talalay method. Data plotted are CI values at 25, 50, 75, and 90% Fa (fraction affected). All the data are expressed as mean±standard deviation (n=3).

FIG. 7 shows that four different drugs, including cytarabine, daunorubicin, GDC-0980 and ARYLATED DIAZENIUMDIOLATE, can be used to perform two-drug combination treatments against acute myeloid leukemia (AML) cells in vitro. Combining cytarabine with GDC-0980 showed the strongest synergistic effect. To improve therapeutic efficacy, N-(2-hydroxypropyl)methacrylamide (HPMA) copolymer-cytarabine and HPMA copolymer-GDC-0980 conjugates were synthesized. Both conjugates had potent cytotoxicity and their combination also showed strong synergism in vitro.

FIG. 8 shows an illustration of the proposed project aiming at the development of new therapeutic strategies for acute myeloid leukemia. Backbone degradable long-circulating HPMA polymer-drug delivery system consists of anti-CD33 mAb fragment as targeting moiety, chemotherapeutic agent cytarabine, and PI3K/mTOR inhibitor GDC-0980. Combination index of Ara-C/GDC showed significant synergy (CI<1) compared with the current clinical standard of Ara-C/daunorubicin.

FIGS. 9A-9E show the development of 2nd generation HPMA copolymer-drug conjugates. A) Development of 2nd generation HPMA copolymer-drug conjugates. Two dithiobenzoate chain transfer agents were linked with lysosomal enzyme cleavable peptide GFLG resulting in a biodegradable RAFT agent, peptide2CTA. This permits one-step synthesis of diblock copolymers. Post-polymerization click reaction produces multiblock HPMA copolymer-drug conjugates with different chain lengths; B) The diblock HPMA copolymer-drug conjugates degraded into half of their initial Mw, indicating the potential to employ diblock conjugates with 100 kDa Mw without impairing their biocompatibility (the degradation products are below the renal threshold) [27]; C) Characterization of 1st- and 2nd generation of gemcitabine conjugates including molecular weight and drug content; D) In vivo fate of 1251-Tyr-2P-drug conjugates following intravenous administration to healthy mice (n=5); E) Tumor growth inhibition in female nude mice after administration of one-dose PTX or on day 0 followed by 3-doses of GEM or its HPMA copolymer conjugates. Note: Traditional HPMA copolymer conjugates are P-PTX and P-GEM; backbone degradable 2nd generation HPMA copolymer conjugates are 2P-PTX and 2P-GEM. In the 2P-PTX+2P-GEM line the error bars are hidden within the experimental points.

FIGS. 10A and 10B show the pharmacokinetics and biodistribution studies of dual-isotope labeled conjugate using SPECT/CT.

FIGS. 11A, 11B, and 11C show the CSC-directed prostate cancer therapeutic system. A). Scheme of HPMA copolymer-drug conjugate system and its function. B). In vivo antitumor activity against PC-3 prostate carcinoma xenografts in nude mice. Treatment groups (n=4): 1) Saline control; 2) P-DTX 10 mg/kg on day 1 3) P-CYP 40 mg/kg twice a week; 4) P-DTX 10 mg/kg on day 1 followed by P-CYP 40 mg/kg twice a week. C). Tumor tissue analysis on percentage of CD133+ cells and prostasphere number after treatment.

FIGS. 12A, 12B, and 12C show the in vivo evaluation of non-hodgkin lymphoma treatment on mouse model. (A). Treatment schedule and Kaplan-Meier plot with indication of numbers of long-term survivors (7 mice per group); (B) Flow cytometry analysis of residual Raji cells in the bone marrow (BM) of the PBS-treated, paralyzed mice (PBS) and the nanomedicine-treated, surviving mice (Cons×3, Prem×3). Bone marrow cells isolated from the femur of mice and Raji cells from culture flasks (upper right panel) were stained with PE-labeled mouse anti-human CD10 and APC-labeled mouse anti-human CD19 antibodies. (C) Quantitative comparison of % Raji cells (human CD10+ CD19+) in the bone marrow of control mice (PBS, n=6) and the nanomedicine-treated mice (Cons×3 and Prem×3, n=7 per group). Statistics was performed by Student's t test of unpaired samples (*: p<0.05). Cons. consecutive administration of Fab′-MORF1 first followed 1 h later by P-MORF2′; Prem premixture was administered.

FIGS. 13A and 13B show a comparison of (A) stability of free drug Ara-C and its HPMA copolymer conjugate in human plasma (the concentration was detected by RP-HPLC on C18 column); (B) growth inhibition of HL-60 cells by free drug Ara-C and its HPMA copolymer conjugate after incubation for 48 h.

FIG. 14 is a table showing the cytotoxicity and combination index of Ara-C, GDC-0980 and their polymer conjugates in HL-60 leukemia cells.

FIGS. 15A and 15B show FMT images of a nu/nu mouse bearing DiR-labeled HL-60 AML cells 24 h after i.v. administration of Cy5-labed anti-human CD33 Ab. Li, liver; Sp, spleen; Ki, kidney; He, heart; St, stomach; Lu, lung; Mu, muscle; Bo, bone; Br, brain.

FIGS. 16A, 16B, 16C, and 16D show super-resolution 3D fluorescent images of endocytosis and drug release in A2780 cells after incubation with model conjugate FITC-P-Cy5 for 4, 8 and 12 h. (A) The lysosome in the cells was pre-stained with LysoTracker Red DND-99 (cyanine); (B) FITC (green) represents polymer chain and (C) Cy5 (red) is cleaved model drug; (D) Merged A, B, and C.

FIG. 17 is a table showing the conjugates to be synthesized and evaluated.

FIG. 18 is a schematic illustration of synthesis of HPMA copolymer-drug conjugates with/without antibody fragments.

FIG. 19 shows the synthesis of HPMA copolymer-drug conjugates followed by sequential dual fluorophore labeling with AF647 for polymer backbone and Cy3B for side-chain drug.

FIG. 20 shows the synthesis of HPMA copolymer-drug conjugates followed by sequential dual isotope labeling with ¹²⁵I for polymer backbone and ¹¹¹In for side-chain drug.

FIG. 21 shows a schematic diagram showing the procedure for nano-fEM.

FIG. 22 shows a schematic diagram showing the investigation of in vivo conjugate-cell interaction

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a HPMA copolymer” includes a plurality of such copolymers, reference to “the HMPA copolymer” is a reference to one or more copolymers and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range¬ from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a HPMA copolymer is disclosed and discussed and a number of modifications that can be made to a number of molecules including the HPMA copolymer are discussed, each and every combination and permutation of the HPMA copolymer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

B. Methods of Treating Acute Myeloid Leukemia (AML) with Combination of AML Therapeutics

Disclosed are methods of treating acute myeloid leukemia (AML) comprising administering an effective amount of at least two of the disclosed compositions. Disclosed are methods of treating AML comprising administering an effective amount of a first AML therapeutic and an effective amount of a second AML therapeutic. In some instances, the first AML therapeutic can be cytarabine, daunorubicin, GDC-0980, arylated diazeniumdiolate, or derivatives thereof. In some instances, the second AML therapeutic can be different from the first AML therapeutic, wherein the second AML therapeutic can be cytarabine, daunorubicin, GDC-0980, arylated diazeniumdiolate, or derivatives thereof.

In some instances of the disclosed methods of treating acute myeloid leukemia (AML) comprising administering an effective amount of a first AML therapeutic and an effective amount of a second AML therapeutic, at least one of the AML therapeutics can be cytarabine. In some instances, at least one of the AML therapeutics can be GDC-0980. For example, disclosed are methods of treating acute myeloid leukemia (AML) comprising administering an effective amount of a first AML therapeutic and an effective amount of a second AML therapeutic, wherein the first AML therapeutic is cytarabine and the second AML therapeutic is GDC-0980.

Disclosed are methods of treating AMLcomprising administering an effective amount of a first AML therapeutic and an effective amount of a second AML therapeutic, wherein the first and second AML therapeutics can provide a synergistic effect.

Disclosed are methods of treating AML comprising administering an effective amount of a first AML therapeutic and an effective amount of a second AML therapeutic, wherein the first and second AML therapeutics can be formulated in a single composition.

Disclosed are methods of treating AML comprising administering an effective amount of a first AML therapeutic and an effective amount of a second AML therapeutic, wherein the first and second AML therapeutics can be formulated in separate compositions.

Disclosed are methods of treating AML comprising administering an effective amount of a first AML therapeutic and an effective amount of a second AML therapeutic, wherein the first and second AML therapeutics are formulated in separate compositions, wherein the first and second AML therapeutics can be administered simultaneously.

Disclosed are methods of treating AML comprising administering an effective amount of a first AML therapeutic and an effective amount of a second AML therapeutic, wherein the first and second AML therapeutics are formulated in separate compositions, wherein the first and second AML therapeutics can be administered consecutively. In some instances, administering consecutively can be administering the compositions comprising the first and second AML therapeutics immediately after one another. In some instances, administering consecutively can be administering the compositions comprising the first and second AML therapeutics within 5, 10, 15, 20, 25, 30, 35, 40, 45, 50, 55, or 60 minutes of one another. In some instances, administering consecutively can be administering the compositions comprising the first and second AML therapeutics within 1, 2, 3, 4, 5, 10, 15, 20, or 24 hours of one another. In some instances, administering consecutively can be administering the compositions comprising the first and second AML therapeutics within 1, 2, 3, 4, 5, 6, or 7 days of one another. In some instances, administering consecutively can be administering the compositions comprising the first and second AML therapeutics within 1, 2, 3, 4, 5, 6, 7, 8, 9, 10, 11, 12, 13, 14, 15, 16, 17, 18, 19, or 20 weeks of one another.

Disclosed are methods of treating AML comprising administering an effective amount of a first AML therapeutic and an effective amount of a second AML therapeutic, wherein if the first AML therapeutic is cytarabine, then the second AML therapeutic is not an anthracycline. Disclosed are methods of treating acute myeloid leukemia (AML) comprising administering an effective amount of a first AML therapeutic and an effective amount of a second AML therapeutic, wherein if the first AML therapeutic is an anthracycline, then the second AML therapeutic is not cytarabine.

Disclosed are methods of treating AML comprising administering an effective amount of a first AML therapeutic and an effective amount of a second AML therapeutic, wherein when the first or second AML therapeutic is cytarabine, the other AML therapeutic is not an anthracycline.

Disclosed are methods of treating AML comprising administering an effective amount of a first AML therapeutic and an effective amount of a second AML therapeutic, wherein the first AML therapeutic is cytarabine, daunorubicin, GDC-0980, arylated diazeniumdiolate, or derivatives thereof, herein the second AML therapeutic is cytarabine, daunorubicin, GDC-0980, arylated diazeniumdiolate, or derivatives thereof, and wherein the first and second AML therapeutics are not cytarabine and anthracycline.

In some instances, the second AML therapeutic is different from the first AML therapeutic.

In some instances, at least one of the AML therapeutics is cytarabine. In some instances, at least one of the AML therapeutics is GDC-0980. Thus, in some instances, the first AML therapeutic is cytarabine and the second AML therapeutic is GDC-0980.

In some instances, the first and second AML therapeutics can provide a synergistic effect.

In some instances, the first and second AML therapeutics can be formulated in a single composition. In some instances, the first and second AML therapeutics can be formulated in separate compositions.

In some instances, the first and second AML therapeutics can be administered simultaneously. In some instances, the first and second AML therapeutics can be administered consecutively. In some instances, administering consecutively can be administering the compositions comprising the first and second AML therapeutics immediately after one another. In some instances, administering consecutively can be administering the compositions comprising the first and second AML therapeutics within 5, 10, 15, 20, 25, 30, 35, 40, 45, 50, 55, or 60 minutes of one another. In some instances, administering consecutively can be administering the compositions comprising the first and second AML therapeutics within 1, 2, 3, 4, 5, 10, 15, 20, or 24 hours of one another. In some instances, administering consecutively can be administering the compositions comprising the first and second AML therapeutics within 1, 2, 3, 4, 5, 6, or 7 days of one another. In some instances, administering consecutively can be administering the compositions comprising the first and second AML therapeutics within 1, 2, 3, 4, 5, 6, 7, 8, 9, 10, 11, 12, 13, 14, 15, 16, 17, 18, 19, or 20 weeks of one another.

In some instances, the disclosed methods comprise administering equivalent dosages of the first and second AML therapeutic. In some instances, the disclosed methods comprise administering different dosages of the first and second AML therapeutic. In some instances, each therapeutic can be administered in a dose of 2 mg/kg-20 mg/kg. For example, GDC-0980 can be administered in 40 mg dose daily or 5-10 mg/kg daily. For example, cytarabine can be administered in 2-6 mg/kg daily. The dosage regimen can be daily, weekly, or monthly. C. Methods of Treating AML with AML Therapeutic Conjugated to a Copolymer

Disclosed are methods of treating AML comprising administering an effective amount of a first AML therapeutic conjugated to a copolymer.

Disclosed are methods of treating AML comprising administering an effective amount of a first AML therapeutic conjugated to a copolymer, further comprising a second AML therapeutic. In some instances, the second AML therapeutic can be conjugated to a copolymer. In some instances, the copolymer conjugated to the second AML therapeutic can be the same or different copolymer conjugated to the first AML therapeutic. In some instances, the first AML therapeutic can be the same or different than the second AML therapeutic.

Traditional copolymers have been used in numerous laboratories worldwide and also in several clinical trials. (See U.S. Pat. No. 5,037,883, which is hereby incorporated by reference in its entirety). For example, N-(2-hydroxypropyl)methacrylamide) (HPMA) copolymers are: (1) biocompatible and have a well-established safety profile; (2) water-soluble and have favorable pharmacokinetics when compared to low molecular weight (free, non-attached) drugs; and (3) possess excellent chemistry flexibility (i.e., monomers containing different side chains can be easily synthesized and incorporated into their structure). However, HPMA polymers are not degradable and the molecular weight of HPMA polymers should be kept below the renal threshold to sustain biocompatibility. This limits the intravascular half-life and accumulation of HPMA polymers in solid tumor via the EPR (enhanced permeability and retention) effect.

To overcome these limitations, a backbone degradable HPMA copolymer carrier was developed. The copolymer carrier can contain enzymatically degradable sequences (i.e., by Cathepsin B, matrix matalloproteinases, etc.) in the main chain (i.e., the polymer backbone) and enzymatically degradable side chains (i.e., for drug release). (See, e.g., U.S. patent application Ser. No. 13/583,270, which is hereby incorporated by reference in its entirety). Upon reaching the lysosomal compartment of cells, the drug is released and concomitantly the polymer carrier is degraded into molecules that are below the renal threshold and can be eliminated from the subject. Thus, diblock or multiblock biodegradable copolymers with increased molecular weight can be produced. This can further enhance the blood circulation time of the Copolymer-AML therapeutic complexes disclosed herein, which is favorable for drug-free macromolecular therapeutics targeting, for example, circulating cancer cells. Furthermore, U.S. Pat. No. 4,062,831 describes a range of water-soluble polymers and U.S. Pat. No. 5,037,883 describes a variety of peptide sequences, both of which are hereby incorporated by reference in their entireties.

Also disclosed are methods of treating AML comprising administering an effective amount of a first AML therapeutic conjugated to HPMA copolymer. In some instances, the first AML therapeutic conjugated to HPMA copolymer is cytarabine.

Disclosed are methods of treating AML comprising administering an effective amount of a first AML therapeutic conjugated to HPMA copolymer, further comprising a second AML therapeutic. In some instances, the second AML therapeutic is GDC-0980. In some instances, the second AML therapeutic can be conjugated to HPMA copolymer. Thus, in some instances, the GDC-0980 is conjugated to a HPMA copolymer.

Disclosed are methods of treating AML comprising administering an effective amount of a first AML therapeutic conjugated to HPMA copolymer, further comprising a second AML therapeutic, wherein the first and second AML therapeutics can provide a synergistic effect.

Disclosed are methods of treating AML comprising administering an effective amount of a first AML therapeutic conjugated to HPMA copolymer, wherein the first AML therapeutic conjugated to HPMA copolymer comprises cytarabine, daunorubicin, GDC-0980, arylated diazeniumdiolate, or derivatives thereof.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises cytarabine, daunorubicin, GDC-0980, arylated diazeniumdiolate, or derivatives thereof.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises cytarabine, daunorubicin, GDC-0980, or arylated diazeniumdiolate, or derivatives thereof, wherein the second AML therapeutic conjugated to HPMA copolymer comprises cytarabine, daunorubicin, GDC-0980, arylated diazeniumdiolate, or derivatives thereof.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises cytarabine, daunorubicin, GDC-0980, arylated diazeniumdiolate, or derivatives thereof, wherein the second AML therapeutic conjugated to HPMA copolymer comprises cytarabine, daunorubicin, GDC-0980, arylated diazeniumdiolate, or derivatives thereof, wherein the second AML therapeutic conjugated to HPMA copolymer is different than the first AML therapeutic conjugated to HPMA copolymer.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises cytarabine, daunorubicin, GDC-0980, arylated diazeniumdiolate, or derivatives thereof, wherein the second AML therapeutic conjugated to HPMA copolymer comprises cytarabine, daunorubicin, GDC-0980, arylated diazeniumdiolate, or derivatives thereof, wherein at least one of the AML therapeutics conjugated to HPMA copolymer is cytarabine.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer can be formulated in a single composition.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer can be formulated in separate compositions.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer can be formulated in separate compositions, wherein the first and second AML therapeutics conjugated to HPMA copolymer are administered simultaneously.

Disclosed are methods of treating AML comprising administering an effective amount of a first AML therapeutic conjugated to HPMA copolymer, further comprising a second AML therapeutic, wherein the first and second AML therapeutics conjugated to HPMA copolymer can be formulated in separate compositions, wherein the first and second AML therapeutics conjugated to HPMA copolymer are administered consecutively.

Disclosed are methods of treating AML comprising administering an effective amount of a first AML therapeutic conjugated to HPMA copolymer, further comprising a second AML therapeutic, wherein at least one of the AML therapeutics conjugated to HPMA copolymer comprise a GFLG linker.

Disclosed are methods of treating AML comprising administering an effective amount of a first AML therapeutic conjugated to HPMA copolymer, further comprising a second AML therapeutic, wherein at least one of the AML therapeutics conjugated to HPMA copolymer further comprises a targeting moiety. In some instances, the first and second AML therapeutics conjugated to HPMA copolymer comprise a targeting moiety. The targeting moiety can be an antibody or fragment thereof. In some instances, the antibody or fragment thereof comprises a Fab region. In some instances, the antibody or fragment thereof can be an anti-CD33 antibody or fragment thereof.

Disclosed are targeting moieties that can be bound, linked, or attached to a copolymer or therapeutic such as, but not limited to, HPMA or cytarabine. In some instances, a targeting moiety can be specific for a particular cell type involved in AML. For example, targeting CD33 or FLT3 on the surface of cells. Cell surface markers that are upregulated on AML cells can be used as a target for the disclosed targeting moieties.

D. Methods of Treating AML with AML Therapeutic Conjugated to a Copolymer Comprising at Least Two Copolymers

Disclosed are methods of treating AML comprising administering an effective amount of a first AML therapeutic conjugated to a copolymer, wherein the first AML therapeutic conjugated to a copolymer comprises a copolymer backbone comprising at least two copolymer segments connected by a degradable peptide sequence. In some instances, the copolymer can be an HPMA copolymer.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence.

Disclosed are methods of treating AML comprising administering an effective amount of a first AML therapeutic conjugated to HPMA copolymer wherein the first AML therapeutic conjugated to HPMA copolymer comprises a HPMA copolymer backbone comprising at least two HPMA copolymer segments connected by a degradable peptide sequence, further comprising a second AML therapeutic, wherein the first AML therapeutic conjugated to HPMA copolymer comprises a HPMA copolymer backbone comprising at least two HPMA copolymer segments connected by a degradable peptide sequence.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises a HPMA copolymer backbone comprising at least two HPMA copolymer segments connected by a degradable peptide sequence, wherein the second AML therapeutic conjugated to HPMA copolymer comprises a HPMA copolymer backbone comprising at least two HPMA copolymer segments connected by degradable peptide sequences.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the first AML therapeutic conjugated to HPMA copolymer comprises at least two AML therapeutics conjugated to the HPMA copolymer backbone.

Disclosed are methods of treating AML comprising administering an effective amount of a first AML therapeutic conjugated to HPMA copolymer, further comprising a second AML therapeutic, wherein the first AML therapeutic conjugated to HPMA copolymer comprises a HPMA copolymer backbone comprising at least two HPMA copolymer segments connected by a degradable peptide sequence, wherein the first AML therapeutic conjugated to HPMA copolymer comprises at least two AML therapeutics conjugated to the HPMA copolymer backbone, wherein the second AML therapeutic conjugated to HPMA copolymer comprises at least two AML therapeutics conjugated to the HPMA copolymer backbone.

In some instances, the AML therapeutics conjugated to HPMA copolymers administered in the disclosed methods can comprise a GFLG linker.

In some instances, the HPMA copolymer backbone of the conjugates administered in the disclosed methods can comprise a GFLG linker.

Disclosed are methods of treating AML comprising administering an effective amount of a first AML therapeutic conjugated to HPMA copolymer, wherein the first AML therapeutic conjugated to HPMA copolymer comprises a HPMA copolymer backbone comprising at least two HPMA copolymer segments connected by a degradable peptide sequence, wherein the HPMA copolymer backbone further comprises a targeting moiety.

Disclosed are methods of treating AML comprising administering an effective amount of a first AML therapeutic conjugated to HPMA copolymer wherein the first AML therapeutic conjugated to HPMA copolymer comprises a HPMA copolymer backbone comprising at least two HPMA copolymer segments connected by a degradable peptide sequence, further comprising a second AML therapeutic, wherein the first AML therapeutic conjugated to HPMA copolymer comprises a HPMA copolymer backbone comprising at least two HPMA copolymer segments connected by a degradable peptide sequence, wherein the HPMA copolymer backbone further comprises a targeting moiety. In some instances the targeting moiety comprises an antibody or fragment thereof. In some instances, the antibody or fragment thereof comprises a Fab region. In some instances, the antibody or fragment thereof can be an anti-CD33 antibody or fragment thereof.

In some instances, the AML therapeutics conjugated to HPMA copolymers administered in the disclosed methods can comprise 2, 3, 4, 5, 6, 7, 8, 9, or 10 HPMA copolymers. In some instances, each HPMA copolymer can be connected via enzymatically degradable peptides.

### E. Compositions

Disclosed herein are compositions comprising a first AML therapeutic and a second AML therapeutic, wherein if the first AML therapeutic is cytarabine, then the second AML therapeutic is not an anthracycline. Disclosed herein are compositions comprising a first AML therapeutic and a second AML therapeutic, wherein if the first AML therapeutic is an anthracycline, then the second AML therapeutic is not cytarabine. Thus, disclosed are compositions comprising a first AML therapeutic and a second AML therapeutic, wherein the first and second AML therapeutics are not the combination of cytarabine and an anthracycline.

Disclosed are compositions comprising a first AML therapeutic and a second AML therapeutic, wherein when the first or second AML therapeutic is cytarabine, the other AML therapeutic is not an anthracycline.

Disclosed are compositions comprising a first and second AML therapeutic, wherein the first AML therapeutic is cytarabine, daunorubicin, GDC-0980, arylated diazeniumdiolate, or derivatives thereof, wherein the second AML therapeutic is cytarabine, daunorubicin, GDC-0980, arylated diazeniumdiolate, or derivatives thereof, and wherein the first and second AML therapeutics are not cytarabine and anthracycline. In some instances, the first AML therapeutic is cytarabine and the second AML therapeutic is GDC-0980.

In some instances, the second AML therapeutic can be different from the first AML therapeutic. In some instances, at least one of the AML therapeutics is cytarabine. In some instances, at least one of the AML therapeutics is GDC-0980.

Disclosed herein are compositions comprising an AML therapeutic conjugated to a copolymer. In some instances, the copolymer can be HPMA. In some instances, the AML therapeutic can be cytarabine, daunorubicin, GDC-0980, arylated diazeniumdiolate, or derivatives thereof.

Disclosed herein are compositions comprising an AML therapeutic conjugated to a copolymer and further comprising a second AML therapeutic. In some instances, the second AML therapeutic can be cytarabine, daunorubicin, GDC-0980, arylated diazeniumdiolate, or derivatives thereof. In some instances, the second AML therapeutic can be conjugated to a copolymer. For example, the copolymer can be HPMA.

### F. Pharmaceutical Compositions

Disclosed are pharmaceutical compositions comprising any of the disclosed compositions herein. For example, in an aspect, a disclosed pharmaceutical composition comprises (i) a first and second AML therapeutic and (ii) a pharmaceutically acceptable carrier.

Disclosed are pharmaceutical compositions comprising a first and second AML therapeutic, wherein the first AML therapeutic is cytarabine, daunorubicin, GDC-0980, arylated diazeniumdiolate, or derivatives thereof, wherein the second AML therapeutic is cytarabine, daunorubicin, GDC-0980, arylated diazeniumdiolate, or derivatives thereof, and wherein the first and second AML therapeutics are not cytarabine and anthracycline.

Disclosed herein are pharmaceutical compositions comprising an AML therapeutic conjugated to a copolymer. For example, in an aspect, a disclosed pharmaceutical composition comprises (i) an AML therapeutic conjugated to a copolymer and (ii) a pharmaceutically acceptable carrier. In an aspect, a disclosed pharmaceutical composition comprises (i) an AML therapeutic conjugated to a HPMA copolymer and (ii) a pharmaceutically acceptable carrier. In an aspect, a disclosed pharmaceutical composition comprises (i) an AML therapeutic conjugated to a copolymer and a targeting moiety and (ii) a pharmaceutically acceptable carrier.

In some instances, any of the disclosed AML therapeutics conjugated to a copolymer can be formulated with a pharmaceutical carrier as a pharmaceutical composition. Thus, disclosed are methods of treating AML comprising administering an effective amount of any of the disclosed pharmaceutical compositions.

In some instances, a disclosed pharmaceutical composition can be administered to a subject in need of treatment of AML. For example, in an aspect, a disclosed pharmaceutical composition can be administered to a subject in need of treatment of a AML.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Tablets and capsules are the preferred oral dosage units whereby solid pharmaceutical carriers are employed. Optionally, tablets can be coated by standard aqueous or nonaqueous techniques. A tablet containing a composition or complex disclosed herein can be prepared by compression or molding, optionally with one or more accessory ingredients or adjuvants. Compressed tablets can be prepared by compressing, in a suitable machine, a disclosed complex of composition in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent. It is understood that the disclosed compositions can be prepared from the disclosed compounds. It is also understood that the disclosed compositions can be employed in the disclosed methods of using.

### G. Kits

Disclosed herein are kits comprising one or more of the compositions described herein.

Disclosed are kits comprising a first AML therapeutic conjugated to a copolymer. In some instances, a disclosed kit can comprise instructions for administering a first AML therapeutic conjugated to a copolymer.

In some instances, disclosed are kits comprising a first AML therapeutic conjugated to a copolymer and a second AML therapeutic. In some instances, the second AML therapeutic can be conjugated to a copolymer. In some instances, the copolymer conjugated to the second AML therapeutic can be the same or different copolymer conjugated to the first AML therapeutic. In some instances, the first AML therapeutic can be the same or different than the second AML therapeutic.

### EXAMPLES

### A. N-(2-Hydroxypropyl)methacrylamide Copolymer-drug Conjugates for Combination Chemotherapy of Acute Myeloid Leukemia

### 1. Introduction

AML is a heterogeneous disease with many molecular mechanisms that lead to resistance to treatment. These include epigenetic dysregulation, gene mutations, overexpression of multidrug resistance genes, abnormal immune function, the presence of chemotherapy-resistant leukemia-initiating cells, and aberrant signaling pathways (i.e., phosphatidylinositol 3-kinase/protein kinase B (PI3K/AKT), mammalian target of rapamycin (mTOR), and Wnt). Due to this molecular heterogeneity, combination of multiple drugs with distinct anticancer mechanisms can offer superior outcomes than single agent therapy. In an effort to find new potent combinations for effective treatment of AML, 4 different agents including cytarabine (CYT), daunorubicin (DAU), GDC-0980 (GDC) and JS-K (JSK) were studied. CYT is an inhibitor of DNA synthesis, while DAU is an anthracycline antibiotic. Both are used as current standard of care for the treatment of AML. Unlike CYT and DAU, GDC and JSK are newly developed drugs that have demonstrated potent antitumor efficacy against a variety of cancer cell lines in vitro and in vivo. GDC is a dual PI3K/mTOR inhibitor and also displays excellent selectivity against other kinases, including DNA-dependent protein kinase, VPS34, c2alpha, and c2beta. GDC is being tested in Phase II clinical trial. JSK is a diazeniumdiolate-based nitric oxide (NO) prodrug that is designed as a substrate for Glutathione S-transferases (GST). GST, which are key phase II detoxification enzymes, are over-expressed in cancer tissues and AML cells. JSK generates little NO spontaneously, but can be activated upon nucleophilic attack by glutathione to release NO which can induce oxidative stress. In addition, JSK was found to modulate Wnt/β-catenin signaling in T-lymphoblastic leukemia cells. Since those 4 drugs have different mechanisms of action, their combination is more likely superior to single agent with respect to potentially targeting different pathways in cancer cells, overcoming drug resistance and maximizing therapeutic efficacy.

Thus, those 4 drugs were combined in pairs and their combined effects on AML cells were investigated in vitro. Results revealed that the combination of CYT and GDC had the strongest synergistic effect. N-(2-hydroxypropyl)methacrylamide (HPMA) copolymer-CYT and HPMA copolymer-GDC conjugates were further synthesized using reversible addition-fragmentation chain transfer (RAFT) copolymerization. In vitro evaluation demonstrated that both conjugates had potent cytotoxicity and strong synergy.

### 2. Materials and Methods

i. Materials and Chemicals

Common reagents were purchased from Sigma-Aldrich (St. Louis, Mo.) and used as received unless otherwise specified. Cytarabine and daunorubicin were purchased from Sigma-Aldrich. GDC-0980 was purchased from Devi Pharma Technology (Suzhou, China). JS-K was synthesized at Richman Chemicals (Lower Gwynedd, Pa.). 2,2′-azobis[2-(2-imidazolin-2-yl)propane]dihydrochloride (VA-044), 4,4′-azobis(4-cyanovaleric Acid) (V-501), and 2,2′-azobis(2,4-dimethyl valeronitrile) (V65) were obtained from Wako Chemicals (Richmond, Va.). 4-cyanopentanoic acid dithiobenzoate, N-(2-hydroxypropyl)methacrylamide (HPMA), N-methacryloylglycylphenylalanylleucylglycine (MA-GFLG-OH), and 3-(N-methacryloylglycylphenylalanylleucylglycyl) thiazolidine-2-thione (MA-GFLG-TT), were synthesized. Human plasma was obtained from University of Utah Blood Bank.

ii. Synthesis of Polymerizable Derivatives of Cytarabine and GDC-0980

N-(methacryloylglycylphenylalanylleucylglycyl) cytarabine (MA-GFLG-CYT) was synthesized by the reaction of MA-GFLG-TT with cytarabine (CYT) in pyridine following the similar procedure described for synthesis of MA-GFLG-Gemcitabine. In brief, MA-GFLG-TT (560 mg, 1 mmol), cytarabine hydrochloride (220 mg, 0.79 mmol) and 6 mg 4-(1,1,3,3-tetramethylbutyl)benzene-1,2-diol (t-octyl pyrocatechol, as inhibitor) were added into an ampoule containing 4 mL pyridine. The system was bubbled with nitrogen for 30 min, then the ampoule was sealed and stirred under 50° C. for 20 h. The solvent was removed by rotary evaporator under vacuum. The crude product was purified by column chromatography (silica gel 60 Å, 200-400 mesh) with gradient elution process from ethyl acetate (EtOAc) to 1:1 EtOAc/acetone and eventually acetone. The white powder was obtained after removal of the solvents with the yield of 300 mg (55.2%). The structure of the monomer was confirmed by MALDI ToF MS ([M+H]+ 686.7), and the purity was verified by HPLC (Agilent 1100 series).

N-(methacryloylglycylphenylalanylleucylglycyl) GDC-0980 (MA-GFLG-GDC) was synthesized by the reaction of MA-GFLG-OH with GDC-0980 in DMF using N-(3-dimethylaminopropyl)-N′-ethylcarbonate (EDC) as coupling agent.[31] After purification on a semi-preparative column (Zorbax 300SB-C18, 250×9.4 mm) using HPLC (Agilent 1100 series), MA-GFLG-GDC was confirmed by MALDI ToF MS ([M+H]+941.4).

iii. Synthesis of HPMA Copolymer-CYT/GDC Conjugates

HPMA copolymer-drug conjugates (P-CYT and P-GDC) were synthesized by the copolymerization of HPMA with MA-GFLG-CYT or MA-GFLG-GDC using 4-cyanopentanoic acid dithiobenzoate as the chain transfer agent (CTA) with the ratio of [Monomer]/[CTA]=560. In the synthesis of P-CYT, HPMA (135 mg, 0.94 mmol) and MA-GFLG-CYT (41 mg, 0.06 mmol) were dissolved in DMSO/H20/0.02% H+ under N2 atmosphere. CTA and V-501 at a molar ratio of 4:1 were added using a syringe. The ampoule was sealed and polymerization was carried out at 70° C. for 10 h. The copolymer was precipitated in acetone, washed with acetone three times, and dried under reduced pressure at room temperature. The dithiobenzoate end group was removed by radical-induced modification with excess of V65. In the synthesis of P-GDC,[31] HPMA (27.8 mg, 0.194 mmol) and MA-GFLG-GDC (5.65 mg, 0.006 mmol) were added into an ampoule. Following three vacuum-nitrogen cycles to remove oxygen, degassed methanol (50 μL), VA-044 solution (0.036 mg in 50 μL) and 4-cyanopentanoic acid dithiobenzoate solution (0.1 mg in 50 μL) in methanol were added via syringe. The mixture was bubbled with nitrogen for 30 min, sealed and polymerized at 40° C. for 22 h. The copolymer was then end-modified in the presence of 40× molar excess of V65 (3.5 mg) in methanol at 50° C. for 4 h. The final product was obtained by precipitation into acetone and purified by dissolution-precipitation in methanol-acetone twice and dried under vacuum. The molecular weight and molecular weight distribution of the conjugates were determined by size-exclusion chromatography (SEC) on an AKTA FPLC system (GE Healthcare, Pittsburgh, Pa.) equipped with miniDAWN and OptilabEX detectors with 50 mM sodium acetate/30% acetonitrile (pH 6.5) as mobile phase. Superose 6 HR10/30 column (GE Healthcare) was used. The drug content in conjugates was determined by enzyme clevage of free drug from polymer side chain GFLG linker using papain.

iv. Stability Evaluation of HPMA Copolymer-Cytarabine Conjugate in Plasma

The stability of free and HPMA copolymer-bound cytarabine in human plasma was evaluated. In brief, both free drug and P-CYT were aliquoted into 100 μL with concentration 100 μg CYT/mL (or CYT equivalent) and incubated at 37oC. At selected time points, 5 μL tetrahydrouridine (THU, 1 mg/mL in DI H2O, used as inhibitor of cytidine deaminase to prevent cytarabine from deamination) was added to a vial containing 100 μL sample. For free drug, 1 mL of mixture of acetonitrile/methanol (9:1) was added to precipitate proteins. The vial was vortex-mixed and then centrifuged at 13,000 g for 10 min at 4° C. The supernatant was carefully transferred into a 2 mL vial that was placed into a 40° C. water bath and bubbled with nitrogen. After organic solvents were removed, the resulting dry residue was dissolved in 0.5 mL DI H2O with 0.1% TFA. The sample was filtered and then 10 μL was injected to an analytical C18 column (Zorbax 300SB, 5 μm, 4.6×250 mm) for HPLC analysis. The amount of intact CYT was calculated based on the area under the curve (AUC) recorded at 268 nm wavelength and calibration curve from a series of standard CYT concentrations. To determine the CYT stability in conjugate, 1-amino-2-propanol (10 μL) was added to the sample prior to plasma precipitation in order to cleave CYT from polymer backbone. Then the sample was examined following the same procedure as described above.

v. Cell Culture

HL-60 human AML cells (ATCC, Manassa, Va.) were maintained at 37° C. in a humidified atmosphere containing 5% CO2 in RPMI-1640 medium (Gibco, Grand Island, N.Y.) supplemented with 2 mM L-glutamine, 10% fetal bovine serum (FBS) and a mixture of antibiotics (100 units/mL penicillin, 0.1 mg/mL streptomycin).

vi. In Vitro Cytotoxicity Study

The cytotoxicity of free drugs (CYT, DAU, GDC, and JSK) and two polymeric conjugates (P-CYT, P-GDC) against HL-60 was measured using the CCK-8 assay (Dojindo, Kumamoto, Japan). The cells were seeded in 96-well plates at a density of 20,000 cells/well in RPMI-1640 media containing 2 mM L-glutamine and 10% FBS. Then, 50 μL media containing the drugs were added. The cells were incubated with free drugs (CYT, DAU, GDC, and JSK) or the polymeric conjugates (P-CYT, P-GDC) at a range of drug concentrations. After 48 h incubation, the number of viable cells was estimated using the CCK-8 kit according to manufacturer's protocol. After the cells were incubated with the reagent at 37° C. for 4 h, the absorbance was measured using a microplate reader at 450 nm (630 nm as reference). Viability of treated cells was calculated as a percentage of the viability of untreated controls.

vii. Combination Effect Analysis

Synergism, additivity or antagonism of the combinations were determined by the Chou-Talalay's method. According to the IC50 values of each drug, the combined molar ratio of CYT, DAU, GDC, and JSK was set as 50:2:50:25. HL-60 cells were treated with combinations of 2 drugs as indicated. Drugs were added with increasing concentrations at a constant molar ratio close to the ratio of the IC50 values for each drug. In the conjugate combination, the molar ratio of CYT to GDC was set as 1:1. A combination index (CI) was determined with the following equation: CI=(D)1/(Dx)1+(D)2/(Dx)2 +α(D)1(D)2/(Dx)1(Dx)2 where (Dx)1 is the dose of agent 1 required to produce x percent effect alone, and (D)1 is the dose of agent 1 required to produce the same x percent effect in combination with (D)2. Similarly, (Dx)2 is the dose of agent 2 required to produce x percent effect alone, and (D)2 is the dose required to produce the same effect in combination with (D)1. The factor a indicates the type of interaction: α=1 for mutually non-exclusive drugs (independent modes of action) in this study. The results are expressed as mutually non-exclusive combination index (CI) values for every fraction affected (Fa), while for the final evaluation we used the averaged CI at 0.25, 0.50, 0.75, and 0.90 Fa, representing relevant growth inhibition values. Here, CI values are plotted against drug effect level Fa. CI values of <0.9 indicate synergy (the smaller the value, the greater the degree of synergy), values >1.1 indicate antagonism and values between 0.9 and 1.1 indicate additive effects. Each experiment was carried out with triplicate cultures for each data point and was repeated independently at least three times.

viii. Cell Cycle Analysis

HL-60 cells (2×10⁵ cells/mL) were seeded in 6-well plates, and treated with drug alone or different combinations of two drugs at the following concentrations for each: CYT=1 μM; DAU=0.04 μM; GDC=1 μM; JSK=0.5 μM. Following 48-h treatment, cells were harvested, fixed and stained with propidium iodide (PI) at room temperature in the dark. Cell cycle analysis was performed by flow cytometry using BD LSR Fortessa machine (BD Biosciences, San Jose, Calif.). Cell percentages in the different phases of cell cycle were analyzed using FlowJo software (Tree star, Ashland, Oreg.).

ix. Statistical Analysis

Data were presented as mean±standard deviation. Statistical analyses were done using a two-tailed unpaired Student's t-test, with p values of <0.01 indicating statistically significant differences.

### 3. Results

i. In Vitro Cytotoxicity

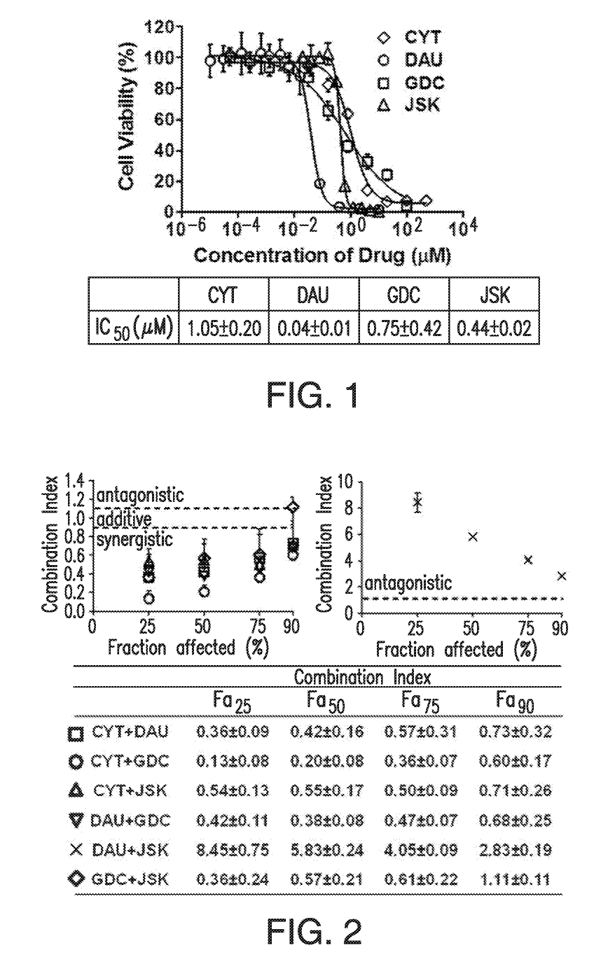

The in vitro cytotoxicity of each individual drug against HL-60 cells was studied. Representative cell-growth inhibition curves and IC₅₀ values are summarized in FIG. 1. All four drugs showed dose-dependent cytotoxicity against HL-60 cells. DAU exhibited the highest in vitro cytotoxicity (IC₅₀=0.04 μM), while the other three drugs had comparable activities (IC50: CYT, 1.05 μM; GDC, 0.75 μM; JSK, 0.44 μM).

ii. Combination Effect

The effect of two-drug combinations on HL-60 cells was also explored. Cells were treated for 48 h with the following combinations at fixed concentration ratios (CYT:DAU:GDC:JSK=50:2:50:25): CYT+DAU, CYT+GDC, CYT+JSK, DAU+GDC, DAU+JSK, and GDC+JSK. Combination Indices (CI) were derived using the Chou and Talalay method. Results are summarized in FIG. 2. CYT had synergistic interactions with all the other 3 drugs, with the strongest synergy observed when it was combined with GDC (FIG. 2). GDC exhibited synergism with JSK (up to 80% Fa level) and DAU. By contrast, the combination of JSK and DAU showed a strong antagonistic effect, with CI values of 8.4, 5.8, 4.1, and 2.8 at the 25, 50, 75, 90% of cells killed level, respectively (FIG. 2).

iii. Cell Cycle Perturbation Following Different Combination Treatments

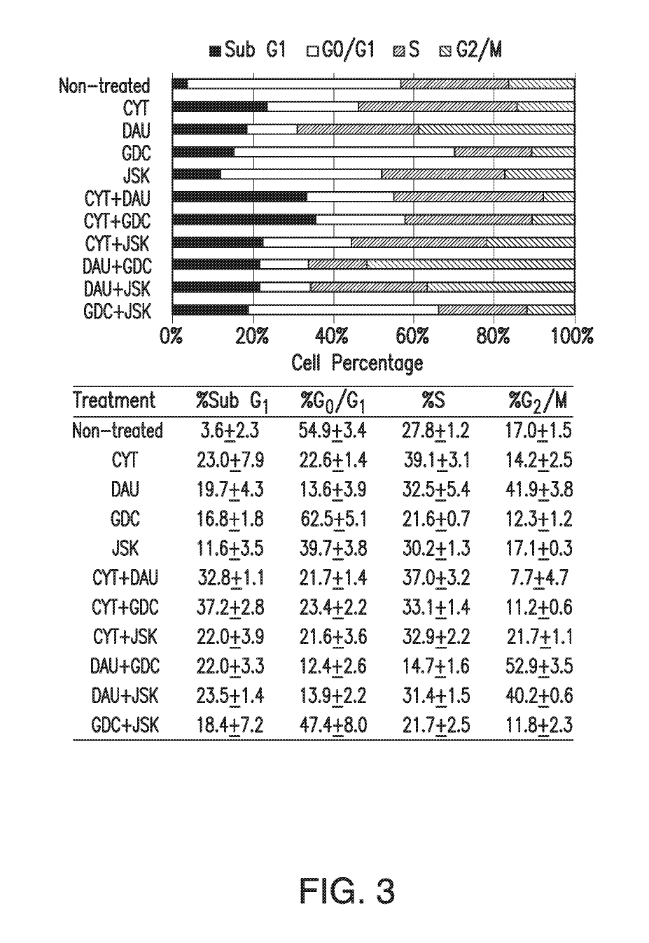

Cell cycle changes of HL-60 cells were further analyzed after exposure to each drug alone, or two-drug combinations. The cell cycle distributions are summarized in FIG. 3. In the single-drug treatment, GDC caused a slight increase in the G0/G1 phase population, whereas CYT, DAU or JSK led to a decrease in the G0/G1 population (FIG. 3). CYT arrested the cells in S phase, while DAU made cells accumulate in the G2/M phase. JSK did not make significant changes in those fractions, indicating induction of apoptosis by JSK at the G0/G1 phase. Combining DAU with GDC or JSK caused a significant increase in the G2/M phase. When combined with any of the other 3 drugs, CYT led to accumulation of cells in the S phase, which was similar to CYT alone.

iv. Synthesis of HPMA Copolymer-drug Conjugates with Improved Solubility/Stability

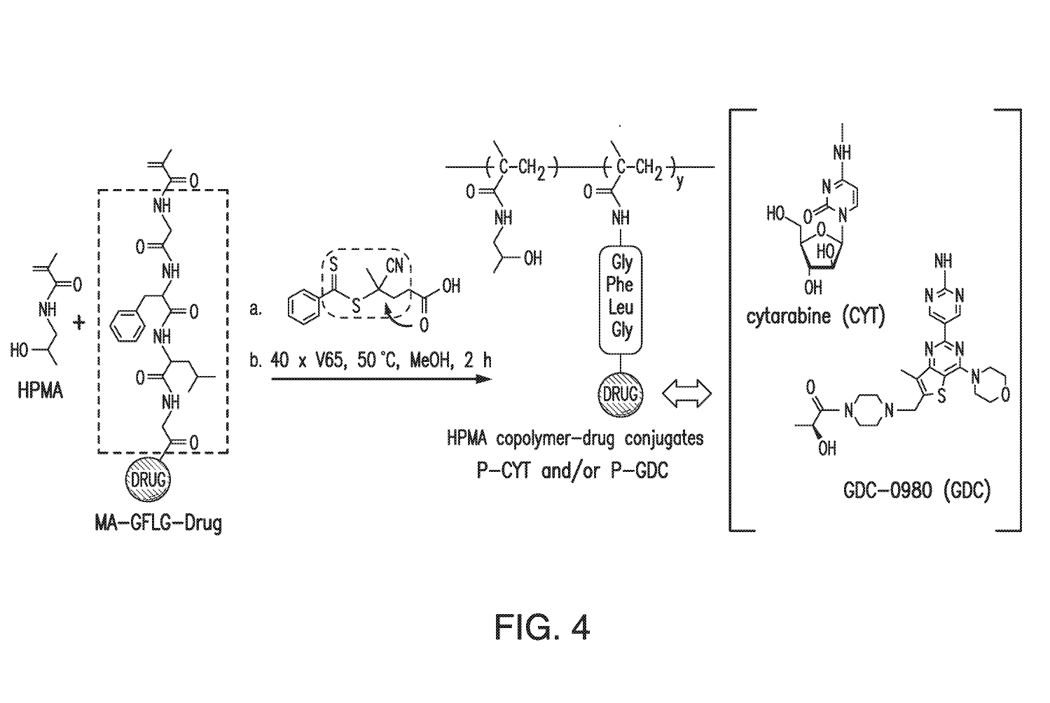

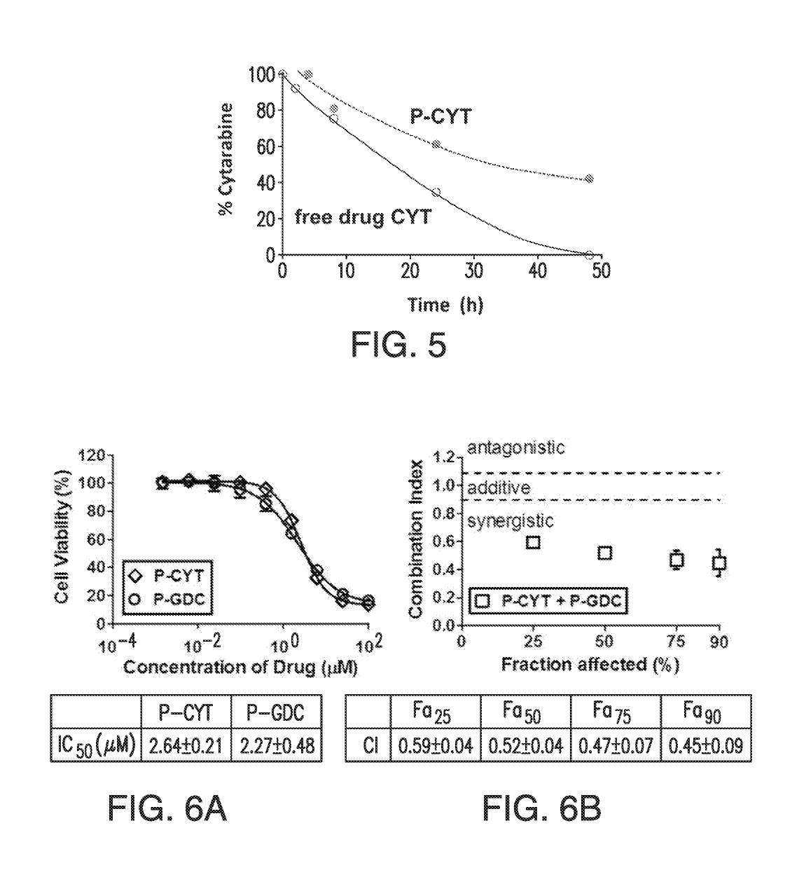

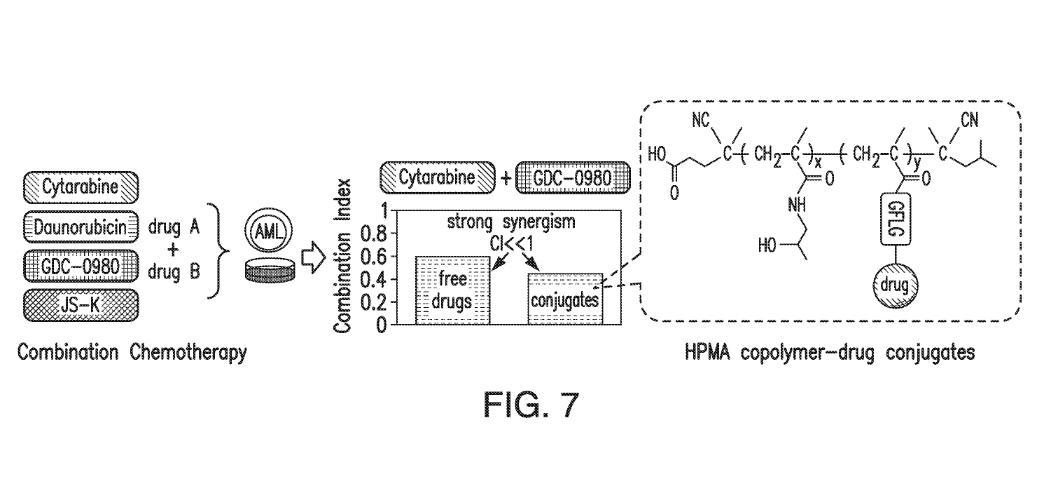

Since the combination of CYT and GDC showed superior anti-leukemic activity compared to the other combinations, the HPMA copolymer-CYT conjugate and HPMA copolymer-GDC conjugates were prepared for polymer-mediated combination chemotherapy. HPMA copolymer-mediated drug delivery can offer some benefits in cancer treatment, including improved bioavailability, prolonged circulation, preferred biodistribution, potential avoid of drug-resistance, and enhanced therapeutic efficacy. The synthesis of HPMA copolymer-CYT and HPMA copolymer-GDC conjugates is depicted in FIG. 4, and the characterization of both conjugates, P-CYT and P-GDC, is listed in Table 1. As a novel class I PI3K/mTOR kinase inhibitor, GDC has been evaluated in various cancer models. Due to poor solubility, GDC was administered orally with 40 mg dose daily in 21 days in a Phase II clinical trial or 5-10 mg/kg daily for 14 days in preclincal studies. Conjugation of GDC to water-soluble HPMA polymer backbone has significantly changed the solubility of the drug. An intravenous injection of P-GDC at dose of 5 mg/kg twice a week in 3 weeks on nude mice bearing PC-3 prostate cancer xenografts has been done. The enhanced anti-tumor activity has been observed. Cytarabine is the most active agent available for the treatment of AML. However, the potency of CYT is limited by its low stability after intravenous administration, and the rapid clearance from the body is due to the metabolism into the inactive and more soluble form by cytidine deaminase. Therefore long-hour infusion and high-dose schedules are always needed. This conjugation strategy clearly demonstrated improved human plasma stability (FIG. 5). After 48 h, all free drug disappeared, whereas there was still close to 50% of the polymer-bound drug present indicating advantage of conjugation of CYT to polymer carrier.

TABLE 1
Characterization of HPMA
copolymer-drug conjugates

| | | | | Drug Content |
|---|---|---|---|---|
| | Mn (kDa) | Mw (kDa) | Mw/Mn | (wt %) |
| P-CYT | 29.9 | 35.0 | 1.17 | 5.4 |
| P-GDC | 45.2 | 47.9 | 1.06 | 4.4 |

v. In Vitro Cytotoxicity and Combination Effect of Conjugates P-CYT and P-GDC

After preparation of the two conjugates (P-CYT and P-GDC), in vitro cell experiments were performed to assess their individual cytotoxicity and their effect in combination. First, HL-60 cells were incubated with individual conjugate for 48 h and analyzed for viability. FIG. 6A shows representative cell-growth inhibition curves and IC₅₀ values. The IC₅₀ values of both conjugates (P-CYT and P-GDC) were 2.64±0.21 μM and 2.27±0.48 μM, respectively, which were higher than the corresponding free drugs. The difference is likely due to different cell uptake mechanisms—endocytosis (conjugates) vs. diffusion (free drugs). As shown in FIG. 6B, the two-conjugate combination exhibited strong synergy, with CI values ranging from 0.45 to 0.59, similar to the free drug combination.

### 4. Discussion

In the United States, leukemia is one of the ten leading causes of cancer deaths and AML is responsible for one third of these deaths. In this study, four antineoplastic agents of different classes for AML treatment, including two traditional drugs (CYT, DAU) and two new ones (GDC, JSK) were investigated. The CI results revealed that CYT and GDC have stronger synergy than the other combinations. The HPMA copolymer-drug conjugates P-CYT and P-GDC were also synthesized. Both conjugates are cytotoxic against HL-60 leukemia cells and their combination exhibited synergism as well.

Current treatment programs for AML are associated with significant toxicity and a high rate of relapse. Due to the molecular and genetic complexity of this disease, single agent therapy aiming at a specific target is unlikely to completely eradicate the malignant clone. Therefore, combination chemotherapy is more likely to induce long term remissions. The current standard approach consisting of a cytarabine/anthracycline combination, followed by either consolidation chemotherapy or allogeneic stem cell transplantation only leads to long-term disease-free survival in a minority of patients. In this study, new two-drug combinations were tested in vitro. On the basis of the CI values (FIG. 2), CYT showed the strongest synergistic interaction with GDC. As a newly developed dual PI3K/mTOR inhibitor, GDC has a potential for future AML treatment, because PI3K/AKT and mTOR signaling pathways are activated in AML: constitutive PI3K activation is detectable in 50% of AML samples whereas mTORC1 is activated in all cases of this disease. In addition, it has been noted that both PI3K and mTOR activation also play an important role for the maintenance and survival of leukemia stem cells (LSCs). AML is composed of biologically distinct leukemic stem, progenitor, and blast populations. LSCs comprise 0.1%-1% of the blasts and are quiescent within bone marrow niches, but are capable of self-renewal. As compared to normal leukemia cells, LSCs have distinct characteristics, such as aberrant surface immunophenotype, dysregulated programs for proliferation, apoptosis, and differentiation, and complex interactions with their surrounding bone marrow microenvironment. All of these factors render LSCs capable of surviving cytotoxic chemotherapy. It has been suggested that LSCs that survive following treatment eventually cause relapse. A high frequency of LSCs at diagnosis is associated with a poor outcome and survival in AML. Although CYT is one of the most effective anti-leukemic drugs, it is ineffective against LSCs. Therefore, combining CYT with other therapeutic agents that specifically target LSCs can be beneficial. The PI3K/mTOR signaling network transmits signals for the maintenance and survival of cancer stem/progenitor cells in AML and other cancers. The PI-103, PI3K/Akt/mTOR inhibitor can inhibit blast cell proliferation and induce mitochondrial apoptosis in the LSCs, indicating that the inhibition of PI3K and mTOR could be used to kill LSCs. As a dual PI3K/mTOR inhibitor, GDC has shown an effective inhibition on PI3K/mTOR signaling pathway in cancer cells. HPMA copolymer-GDC conjugates can effectively inhibit CD133+ prostate cancer stem/progenitor cells at low concentrations. GDC can possess potent anti-LSCs activity. Consequently, the combination of CYT and GDC can be active against LSCs.

Effective drug delivery is essential for the optimal use of drug combinations. In the clinic, CYT is generally given at either high intravenous doses or by continuous infusion, because of its short plasma half-life and low stability. Recently, several polymer-based formulations have been developed to improve the delivery of CYT and DAU. For example, Elacytarabine, a conjugated form of CYT to the lipid moiety elaidic acid, is currently under investigation in a randomized trial for relapsed AML patients. Another new formulation, CPX-351 is a liposomal carrier containing CYT and DAU in a fixed molar ratio (5:1), which is also being tested in AML patients. In this study, the HPMA copolymer was employed as a carrier for CYT and GDC, because the HPMA copolymer-based drug delivery systems have several advantages, including enhanced drug bioavailability, improved pharmacokinetics, increased drug accumulation at the tumor site, decreased non-specific toxicity, and controlled drug release. In particular, conjugation of CYT to HPMA polymer carrier prevented CYT from degradation into inactive metabolite and increased its plasma half-life (FIG. 5). These properties are most important factors that help improve the therapeutic index. Moreover, the HPMA copolymer drug conjugates can overcome drug resistance. Overexpression of the P-glycoprotein (P-gp) and multidrug resistance (MDR)-associated proteins is a key factor contributing to treatment failure in AML by reducing intracellular accumulation of cytotoxic drugs. GDC and several commonly used drugs in AML, including daunorubicin, mitoxantrone, and etoposide are substrates for the P-gp. HPMA copolymer conjugates can release drugs intracellularly and circumvent the effect of membrane efflux pumps such as P-gp.

### 5. Conclusions

In the two-drug combinations tested, strong synergistic interactions were found between commonly used drugs (CYT, DAU) and newly developed agents (GDC, JSK). In particular, the combination of CYT and GDC showed the strongest synergism. HPMA copolymer-drug conjugates provide significant pharmacologic advantages.

### B. Targeting Long-Circulating Macromolecular Therapeutics to AML Cells

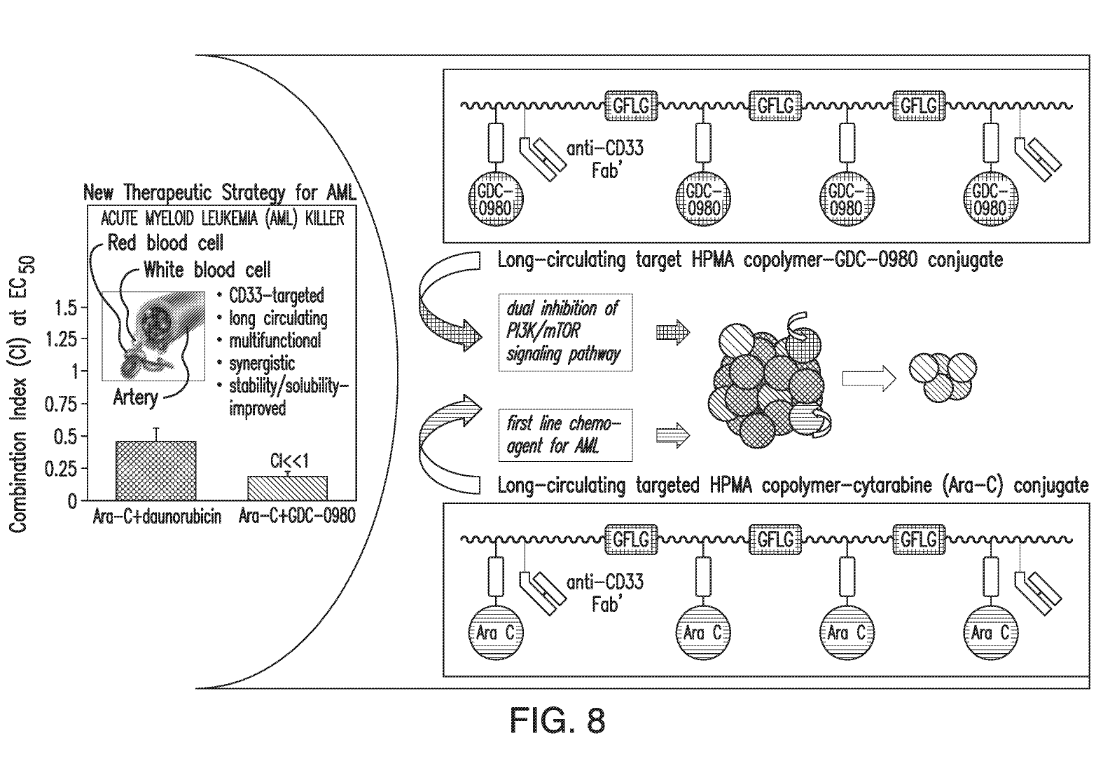

Acute myeloid leukemia (AML) is the most common acute leukemia in adults with only 20 to 30% survivors. The mainstay of therapy has been cytarabine (Ara-C) and anthracyclines. Since the 1970s, no new agent has had a major impact on the disease except for acute promyelocytic leukemia. Even allogeneic stem cell transplant cures only about 50% of eligible patients. The need for new effective agents is therefore evident. A new strategy to treat AML is proposed herein (FIG. 8). The distinct features include: a) Targeting long-circulating macromolecular therapeutics to AML cells; b) Development of synergistic combinations of new agents; c) Non-invasive evaluation of treatment efficacy using molecular imaging tools.

This new therapy provides the following: a) new concept for AML therapeutics, namely combination of two polymer synergistic drugs; b) new targeted agents for AML using a target (CD33) and antibody (hP67.6) that have been proven to be clinically valid; c) development of new AML-specific nanomedicines based on high molecular weight long circulating polymeric carriers containing enzymatically degradable bonds in the main chain; d) designing a high-sensitivity approach suitable for detection of minimal residual disease (MRD).

AML, Leukemic Stem Cells (LSC), MRD, and CD33. The goal of AML therapy is an initial complete remission (CR) (undetectable leukemia and restoration of normal hematopoiesis) using synergistic drug combinations, classically Ara-C and an anthracycline. This approach has significant toxicity. Most, if not all patients in CR harbor residual disease, necessitating further treatment aiming at cure. Except for pediatric patients and promyelocytic leukemia, MRD detection is still not used to guide therapy. The main problem with AML treatment is the high relapse rate. Relapse is due to persistence of resistant LSCs.

LSCs from different patients are heterogeneous. AML results from multiple genetic events and could arise at the level of multipotent hematopoietic stem cells (HSCs) or at the level of committed precursor cells. Like their normal counterpart, LSCs undergo self-renewal and are generally in a quiescent state, thus resisting killing by cell cycle-dependent agents like Ara-C. Drugs expected to target LSCs should have at least one of the following attributes: cell cycle-independence, inhibition of NF-κ3 signaling, induction of oxidative stress, or inhibition of self-renewal mechanisms such as the Wnt/β-catenin pathway.

MRD, defined as the existence of leukemic cells after chemotherapy treatment and thought to be responsible for relapse, is important in evaluation of treatment effectiveness. Currently real-time quantitative polymerase chain reaction and multiparameter flow cytometry are the two most commonly used techniques. Development of non-invasive detection of MRD with high specificity and sensitivity will improve clinical decision-making.

CD33 is a 67 kDa glycosylated transmembrane protein that belongs to the sialic acid-binding immunoglogulin-like lectin family. It is expressed on the surface of normal myeloid cells and on the majority of AML blasts. It is not expressed at high levels by normal HSCs. Indeed, leukemic stem cells within the CD34+/CD38− compartment were recently shown to have much higher CD33 levels than healthy donor stem cells. When engaged by an antibody, CD33 internalizes into the cell, making it an attractive target for drug delivery. Gemtuzumab ozogamicin, an anti-CD33 antibody conjugated to calicheamicin, is quite active in AML. However, calicheamicin is a substrate for the P-glycoprotein and GO can be hepatotoxic.

Cytarabine (Ara-C). Ara-C is one of the most active agents for the treatment of AML. It gets phosphorylated to its active metabolite (Ara-C triphosphate), which functions as a DNA polymerase inhibitor. Ara-C is used either as a single agent or in combinations with other anticancer drugs at different doses/schedules in very potent combinations. However, high-doses cause toxicity and high relapse rates due to drug-resistance; these are the key limiting factors for treatment. A delivery system that targets Ara-C to AML cells can significantly improve Ara-C's pharmacologic and toxicologic profiles.

Anti-leukemic effect of PI3K/mTOR dual inhibitor and GDC-0980. The phosphatidylinositol 3-kinase (PI3K)/mammalian target of rapamycin (mTOR) signaling network is aberrantly activated in many solid tumors. Its signals are vital for cancer cell survival, proliferation and drug-resistance. PI3K/Akt/mTOR is constitutively activated in the majority of AML patients and is associated with shorter disease-free survival. PI3K/mTOR signaling is active not only in bulk AML cells, but also in LSCs. mTOR or PI3K/mTOR dual inhibitors deplete LSCs and restore normal hematopoiesis. Several PI3K, mTOR, or PI3K/mTOR dual inhibitors are being investigated in patients with AML. In the case of AML, the mTOR pathway is usually activated independently of the activated PI3K/Akt cascade; in addition, inhibiting only mTOR could overactivate the PI3K/Akt pathway through a feedback mechanism. Thus, dual PI3K/mTOR inhibitors are advantageous. Among dual PI3K/mTOR inhibitors, GDC-0980 is potent in inhibiting both Class I PI3 kinase and mTORC1/2 (Complex 1/2). It is in clinical trials for solid tumors and non-Hodgkin's lymphoma. Based on its mechanism of action, it could be of unique benefit in AML.

Macromolecular therapeutics and N-(2-hydroxypropyl)methacrylamide (HPMA) copolymer carrier for anticancer drug delivery. The concept of polymer-drug conjugates was developed to address sub-optimal bioactivity and non-specificity of low-molecular weight drugs. While drugs can penetrate into all cell types via diffusion, attachment of these drugs to polymer backbone limits the cellular uptake to the endocytic route, changes their pharmacokinetics, resulting in decreased adverse effects.

For example, doxorubicin (DOX) is cardiotoxic; its maximum tolerated dose (MTD) in humans is 60-80 mg/m2, whereas the MTD of HPMA copolymer-DOX conjugate (P-DOX; P is the HPMA copolymer backbone) is 320 mg/m2 (in DOX equivalent) mainly due to less effective accumulation of P-DOX in heart tissue [20]. Indeed, following P(GFLG)-DOX administration in mice, only low concentrations of DOX were detected in brain, liver, kidney, lung, spleen, heart and other organs. As expected, enhanced accumulation of P(GFLG)-DOX in the tumor was observed. Macromolecular therapeutics have the potential to overcome the efflux pump type of multidrug resistance. The anticancer activities of free DOX and HPMA copolymer-bound DOX [P(GFLG)-DOX] were studied in mouse models of DOX sensitive (A2780) and DOX resistant (A2780/AD) human ovarian carcinoma xenografts [21]. Free DOX was effective only in sensitive tumors, decreasing tumor size about three times, while P(GFLG)-DOX decreased tumor size 28 and 18 times in the sensitive and resistant tumors, respectively.

HPMA copolymers as drug carriers were invented in the 70s. Drugs are attached to polymer carriers via the enzyme-sensitive oligopeptide GFLG. The stability of the conjugates in blood plasma/serum and degradability by lysosomal enzymes such as cathepsin B have been thoroughly investigated. The P-DOXs with/without targeting moiety (PK2/PK1) have been tested in Phase I and Phase II clinical trials to treat various solid tumors such as liver, breast, lung and colon cancers.

Molecular weight (Mw) and Mw distribution are important factors in the design of effective macromolecular therapeutics. To take full advantage of increased systemic circulation time and increased tumor accumulation via the EPR effect, HPMA copolymers must be large enough to evade renal filtration (i.e., greater than 50 kDa). However, to avoid non-degradable drug carrier accumulation in various organs and compromising biocompatibility, the Mw of 1st generation HPMA copolymer-drug conjugates used in clinical trials was ˜28 kDa. This lowered the retention time of the conjugates in the circulation and decreased their efficacy. As a result, no product has received approval at this point.

Long-circulating multiblock backbone degradable HPMA copolymers containing enzyme-sensitive GFLG linkers have been synthesized by click coupling of RAFT-generated telechelic HPMA copolymer-drug conjugates. In several animal models substantially augmented therapeutic efficacy of the 2nd generation conjugates have been demonstrated when compared to the 1st generation conjugates.

Targeted macromolecular (polymer) therapeutics and combination therapy. Targeted polymer-drug conjugates can be prepared by attaching a ligand that complements a receptor/antigen on the target cell, thus increasing the specificity of the delivery system. The concept of using combination therapy with water-soluble polymer-bound drugs has been developed. In vivo combination chemotherapy and photodynamic therapy (PDT) studies on two cancer models, Neuro 2A neuroblastoma in A/J mice and human ovarian carcinoma heterotransplanted in nude mice, demonstrated that combination therapy produced tumor cures which could not be obtained with either therapy alone. The OV-TL16 monoclonal antibody recognizes the OA-3 (CD47) antigen expressed on the surface of OVCAR-3 cells and the majority of human ovarian carcinomas. OV-TL16 and its Fab′ fragment have been used to target HPMA copolymer-drug conjugates to human ovarian carcinomas with high efficacy. Comparison of non-targeted and OV-TL16-targeted HPMA copolymer-DOX and HPMA copolymer-mesochlorin e₆ conjugates in combination chemotherapy and photodynamic therapy in OVCAR-3 xenografts demonstrated the advantage of targeted combination treatment. The immunoconjugates preferentially accumulated in human ovarian carcinoma OVCAR-3 xenografts with increased efficacy when compared with non-targeted conjugates.

Multimodality Imaging. Over the past decade, imaging techniques have been widely used to facilitate the development of nanomedicines. For example, single-photon emission computed tomography/computed tomography (SPECT/CT), is rapidly growing in both pre-clinical and clinical studies, because it can concurrently investigate two different radiotracers in a specific organ to allow the visualization of different molecular functions under the same physiological and physical conditions. In addition, several nano-scale resolution fluorescence technologies (˜20 nm) were recently developed, including photoactivated localization microscopy (PALM) and stochastic optical reconstruction microscopy (STORM). These state-of-the-art “nanoscopes” show a 10-fold improvement over the resolution of conventional fluorescence microscopy, allowing the separation of fluorophores closer than the diffraction limit. Optical imaging techniques, including fluorescence imaging and bioluminescence imaging, play an important role in preclinical research as advances in photonic technology and reporter strategies have led to widespread exploration of biological processes in vivo. One of the most recent technological evolutions has been the development of fluorescence tomography for visualization at the whole animal or tissue level. For example, fluorescence molecular tomography (FMT) technology not only can provide non-invasive, whole-body, deep-tissue imaging in small animal models, but also enable 3D quantitative determination of fluorochrome distribution in tissues of live animals. In this study, multiple imaging modalities can be integrated to explore the optimal schedule/dose of the combination targeting system, to monitor the therapeutic efficacy, and to develop high sensitivity Gaussia-lucierease (Gluc)-based bioluminescence imaging for MRD detection.

### 1. Results

i. Design, Synthesis and Evaluation of Biodegradable Long-Circulating HPMA Copolymer-Drug Conjugates

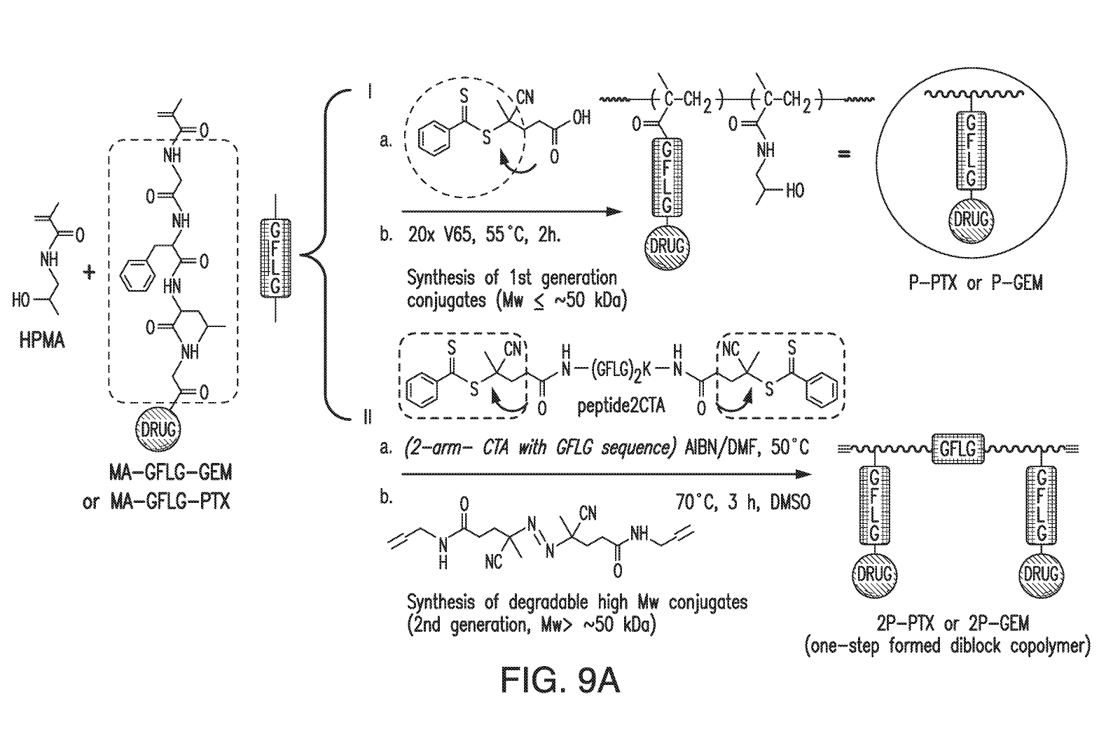

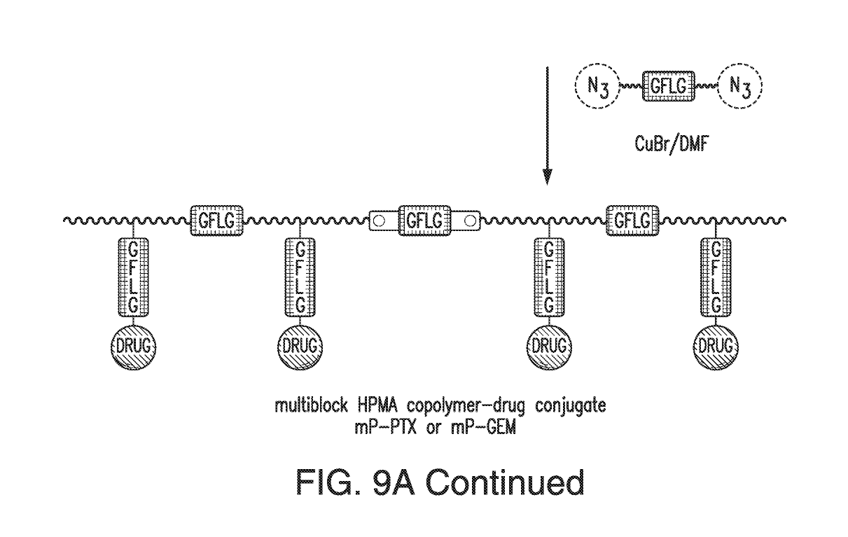

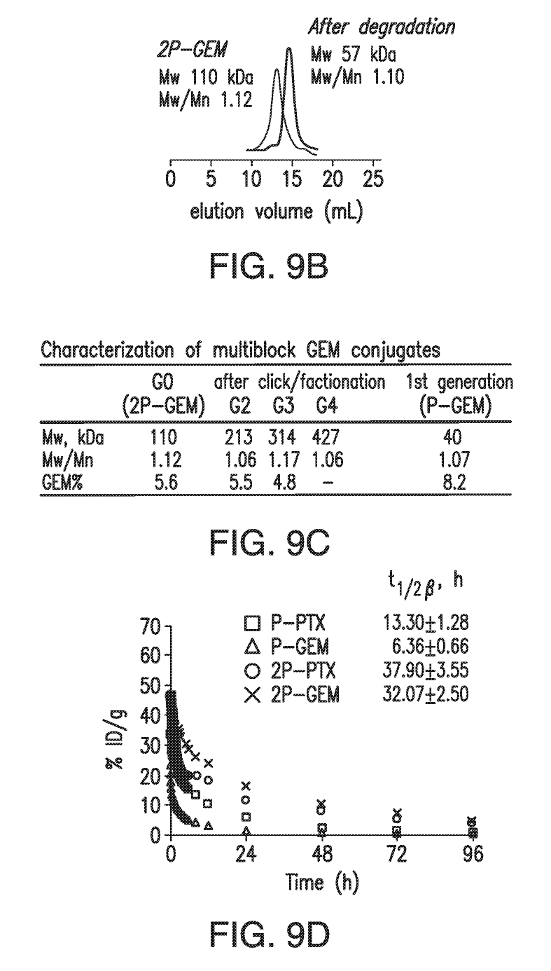

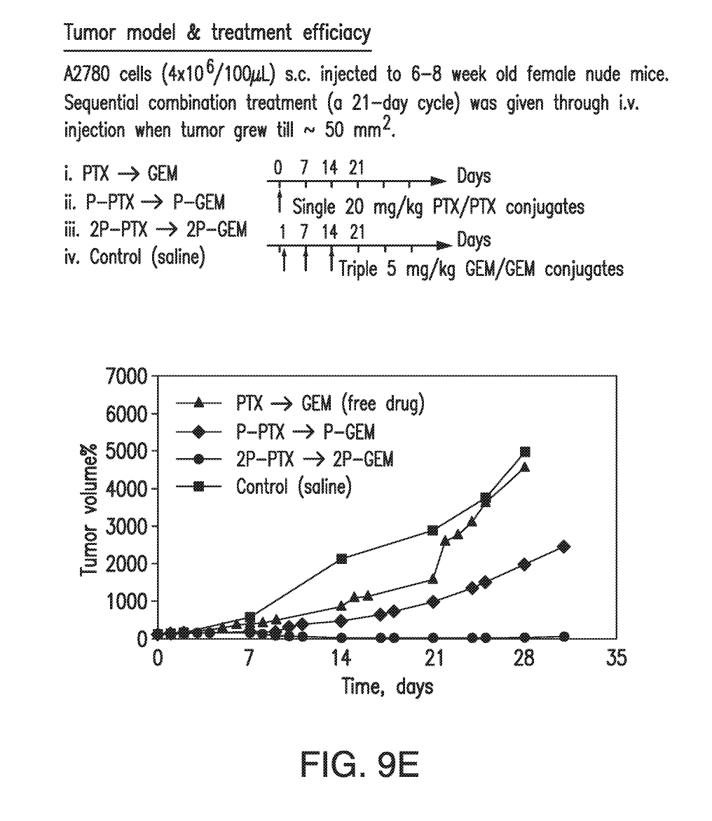

The hallmark of the 2nd generation conjugates is the multiblock structure composed of alternating HPMA copolymer blocks and enzyme-cleavable oligopeptide segments with tailored biodegradability (FIG. 9). This permits use of high Mw long-circulating conjugates without impairing biocompatibility. Also, utilization of polymerizable drug derivatives and controlled living polymerization chemistry results in narrow distribution of Mw and minimal heterogeneity in chemical composition of the conjugates.

The pharmacokinetic behavior of 1st generation gemcitabine (GEM) and paclitaxel (PTX) conjugates (P-PTX and P-GEM) and diblock backbone degradable conjugates (2P-PTX and 2P-GEM), an example of 2nd generation conjugates, were determined by radiolabeling pendant Tyr in the polymer carrier with ¹²⁵I. The blood radioactivity-time profiles of the four conjugates in mice are illustrated in FIG. 9D. A two-compartmental model was used to analyze blood pharmacokinetics. It was confirmed that PK parameters of diblock conjugates were more favorable than those of lower Mw conjugates. Treatment of A2780 human ovarian carcinoma xenografts with GEM and PTX is shown in FIG. 9E. Compared to free drugs and the 1st generation HPMA copolymer-drug conjugates, the 2nd generation backbone degradable HPMA copolymer conjugates have significantly increased antitumor activity. Long retention time of drugs in the circulation is crucial. Increased circulation time and enhanced tumor accumulation of high Mw backbone degradable HPMA copolymer-drug conjugates increase exposure of tumor cells to the drugs (drug concentration×time of exposure).

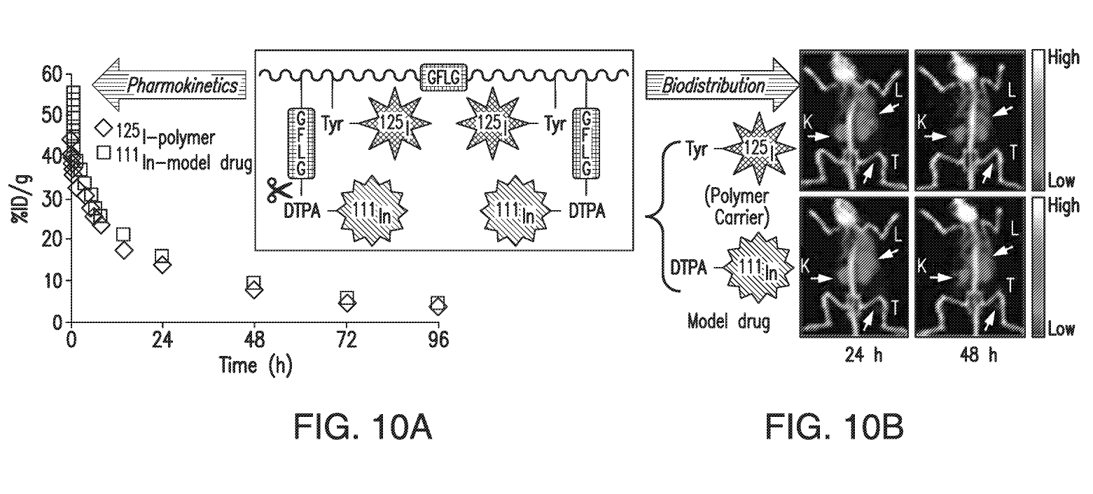

To support this conclusion, diethylene triamine pentaacetic acid (DTPA) was used as a model drug to synthesize dual-labeled HPMA copolymer conjugates and determined the (separate) fates of HPMA copolymer carrier and DTPA in vivo (FIG. 10). ¹²⁵I labeled polymer backbone and side chains terminated DTPA with complexed 111In were monitored separately after i.v. injection to healthy female nude mice (6-8 weeks old; n=5). At predetermined time intervals, blood samples (10 μL) were taken from the tail vein. The radioactivity of ¹²⁵I and ¹¹¹In was counted in channels with windows set for 15-85 keV and 237-257 keV, respectively. Results showed that the payload In-DTPA had similar pharmacokinetics profile as ¹²⁵I-labeled polymeric carrier in mice (FIG. 10A). In addition, the biodistribution of the polymer carrier and the pendant model drug was evaluated by SPECT/CT imaging. The above dual-labeled HPMA copolymer conjugate was i.v. injected to female nude mice bearing A2780 human ovarian carcinoma xenografts (6-8 weeks old; n=5). At 48 h after injection, the tumor uptake of ¹¹¹In-DTPA and ¹²⁵I-Tyr-Polymer reached 4.08% ID/g and 4.57% ID/g, respectively, whereas the 1st generation model conjugate only showed 0.77% for ¹¹¹In and 0.53% for ¹²⁵I. The results indicate that: a) the linker GFLG between drug model and polymeric carrier is stable in blood circulation during transport; b) backbone degradable HPMA copolymer carrier can effectively deliver drugs to the tumor.

ii. Combination Treatment Targeting Both Differentiated and Cancer Stem Cells (CSCs) Results in Superior Long-Term Tumor Growth Inhibition

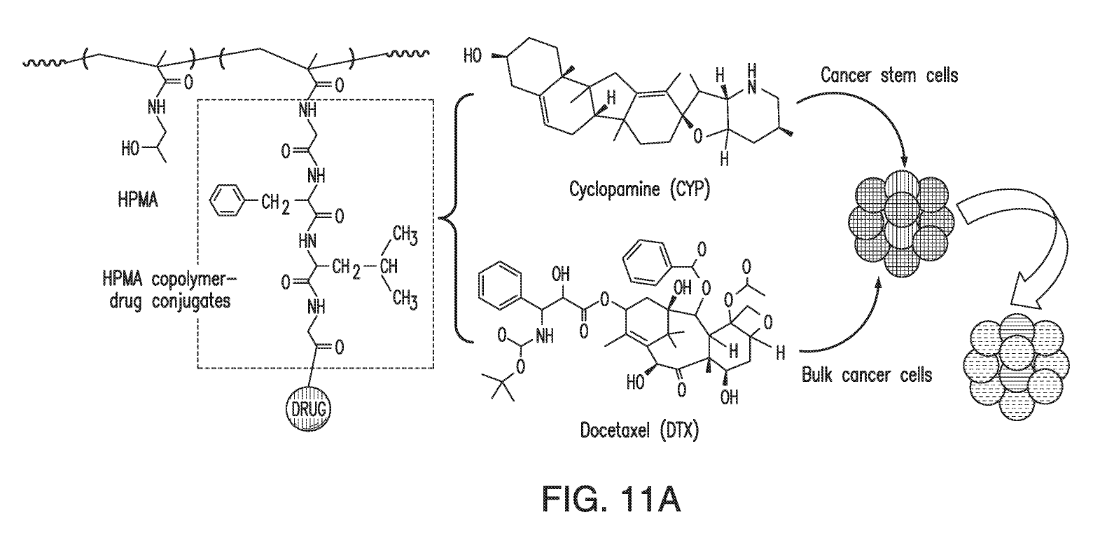

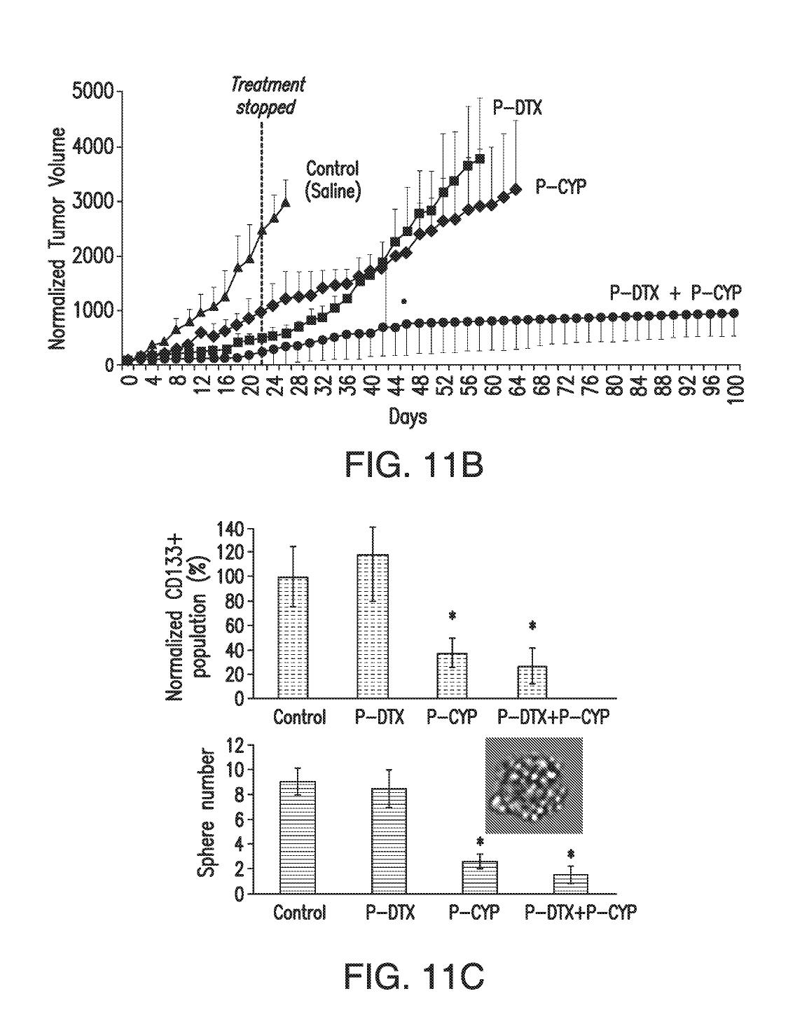

A small, stem-like cell population in several human cancers is not sensitive to standard therapy. It is able to self-renew and is crucial for tumor relapse. The ideal anticancer regime should be able to eradicate differentiated cancer cells to reduce tumor mass and, simultaneously, to eliminate the CSC population. A therapeutic strategy has been developed for prostate cancer using a combination system consisting of cyclopamine (CYP) conjugate and docetaxel (DTX) conjugate. DTX is a traditional first-line chemotherapeutic agent for advanced prostate cancer, whereas CYP has been reported to inhibit the growth of prostate CSCs by inhibiting the hedgehog signaling pathway. This combination nanomedicine was evaluated on PC-3 tumor bearing nu/nu mice (FIG. 11). The treatment was discontinued after 21 days but tumor growth was continuously monitored for a longer period. As shown in FIG. 11B, tumors in P-DTX group started to regrow faster on average after stopping the treatment; tumors in P-CYP group continue to grow progressively; strikingly, the combination of P-CYP and P-DTX showed the most persistent tumor growth inhibition, leading to the longest mice survival on average. Residual tumors were examined after sacrificing the mice. CD133 expression level and sphere-forming capacity, which are used as a measure of stem cell properties, were analyzed. The combination group showed significantly decreased sphere formation and low CD133 expression. This observation, together with the tumor growth inhibition, demonstrates the ability of the new combination strategy to eradicate all cancer cells, including CSCs.

iii. CD20-Targeted HPMA Copolymer-Based System for the Treatment of Non-Hodgkin Lymphoma (NHL)

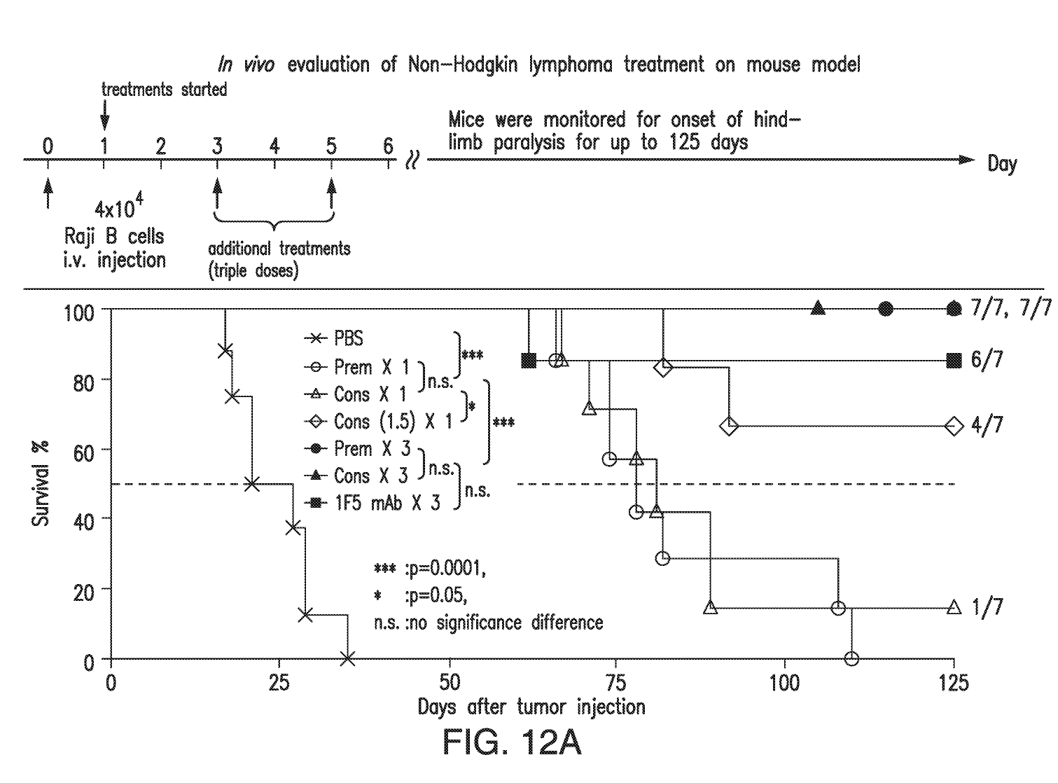

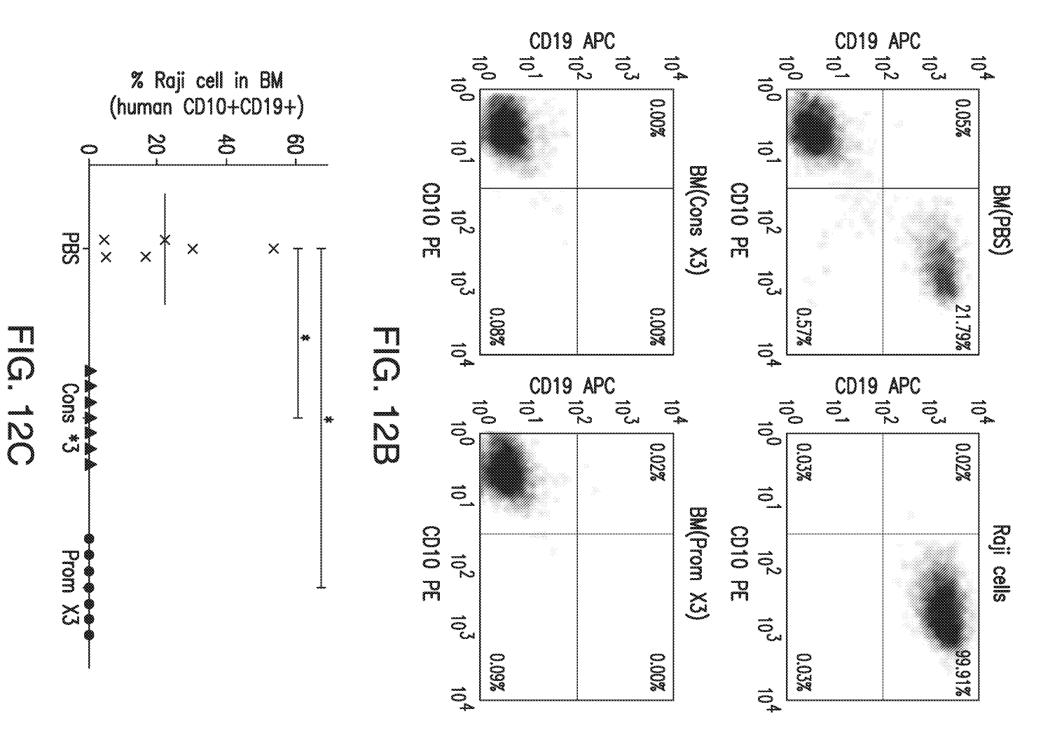

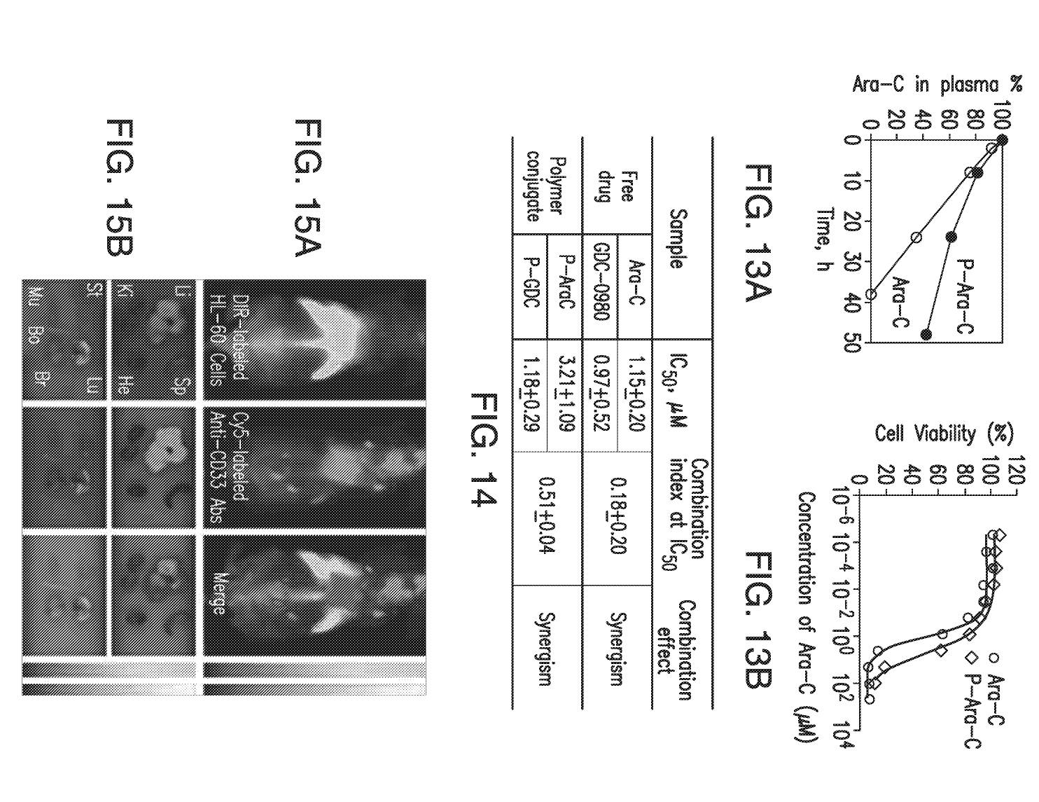

HPMA copolymer conjugates have been used not only for the treatment of solid tumors, but also for blood cancer. A HPMA copolymer-centered biorecognition system targeted to CD20 for B-cell lymphoid malignancy treatment was developed. It is based on the biorecognition of two complementary biological motifs, coiled-coil forming peptides (i.e. CCE/CCK) or oligonucleotides (i.e. MORF1/MORF2), at the surface of B-cells. One peptide or oligonucleotide is conjugated to the anti-CD20 Fab′ fragment. The complementary moiety is conjugated in multiple grafts to polyHPMA. Their biorecognition results in crosslinking of CD20 antigens at the surface of CD20+ malignant B-cells and initiation of apoptosis. Treatment of systemically disseminated CD20+ Raji B cell lymphoma in C.B.-17 SCID mice with Fab′-MORF1 and P-MORF2 led to long-term survivors (125 days, FIG. 12A). Eradication of Raji cells after treatment was further confirmed by flow cytometry (FIG. 12B,C), MRI and histology.

iv. Preliminary Evaluation of the Novel Combination Nanomedicine Consisting of HPMA Copolymer-Cytarabine Conjugate and HPMA Copolymer—GDC-0980 Conjugate

a. Stabilization of Labile Drugs by Conjugation to Polymeric Carrier.

Cytarabine (Ara-C) has a very short half-life in vivo due to rapid loss via degradation in the blood stream and fast renal excretion. It is therefore necessary to administer the drug either by continuous infusion or bolus high doses in the clinics. HPMA copolymer-Ara-C conjugate was synthesized with improved stability (FIG. 13A) without compromising efficacy (FIG. 13B). These results show the translational potential of the conjugate.

b. Synergistic Effect of Proposed Drugs on Leukemia Cells.

We have synthesized HPMA copolymer conjugates containing Ara-C and the PI3K/mTOR dual inhibitor GDC-0980, using the procedure described in FIG. 9. The cytotoxicity and the interaction of free drugs and their polymer conjugates toward leukemia cells were evaluated. Luciferase-expressing HL-60 leukemia cells were incubated with the individual drugs or HPMA copolymer conjugates in different combinations for 48 h with the dose ratio based on the IC₅₀ value of each individual drug. Drug interactions were evaluated using the Combination Index (CI) determined by the Chou-Talalay median effect analysis. Values of CI<1 indicate synergism; CI=1 indicates additivity; and CI>1 indicates antagonism. As shown in FIG. 14 there was significant synergy between the 2 agents.

v. Imaging Studies

a. Evaluation of Targeting Specificity Using Fluorescence Molecular Tomography (FMT).

To evaluate the targeting specificity of the anti-CD33 antibody for the development of non-invasive MRD diagnostics, HL-60 AML cells were labeled with DiR (lipophilic carbocyanine) and injected into a nu/nu mouse via the tail vein. After 24 h, the mouse received an i.v. injection of Cy5-labeled anti-human CD33 antibody (mAb-Cy5). Another 24 h later, the mouse was scanned using FMT. Most HL-60 cells distributed in the liver, spleen, spine, femora, and lung (FIG. 15A). The mAb-Cy5 accumulated in “hot” tissues where leukemia cells accumulated. Major organs were then harvested and analyzed. Ex vivo fluorescence images showed co-localization of DiR-labeled HL-60 cells and mAb-Cy5, thus confirming the targeting efficiency of mAb-Cy5 (FIG. 15B).

b. Internalization and Subcellular Fate of the Conjugates.

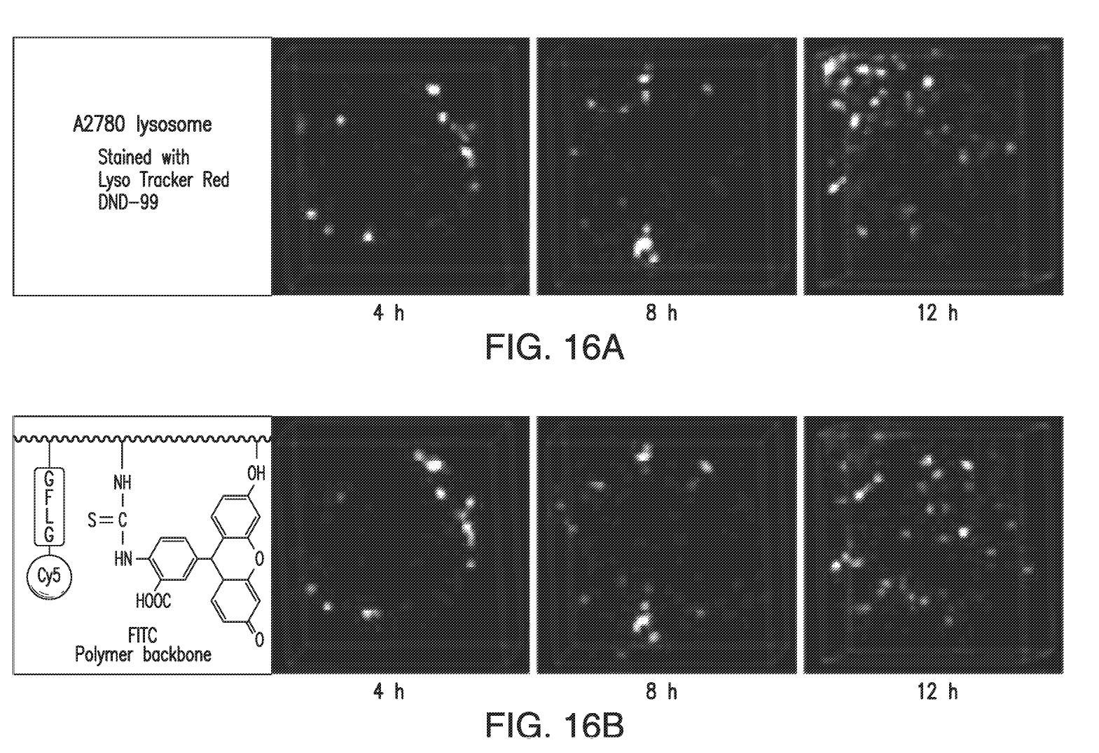

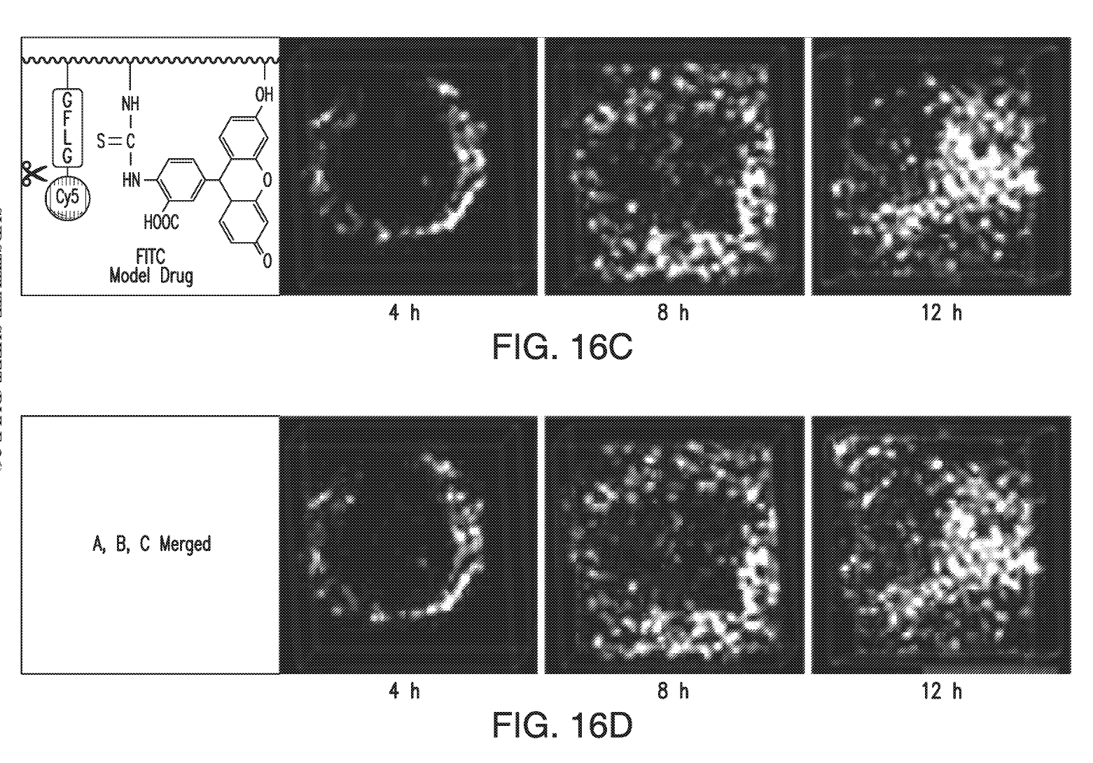

A dual-fluorophore labeled model conjugate FITC-P-Cy5 (FITC labeled HPMA copolymer containing Cy5 as a drug model) and super-resolution fluorescence imaging were used to evaluate internalization and drug release at the single cell level. A2780 cells were visualized under a 3D super-resolution Vutara SR-200 fluorescence microscope equipped with a FITC filter (wavelength 495/519 nm), a Cy5 filter (wavelength 650/670 nm), and a Red DND-99 filter (wavelength 557/590 nm). Images were analyzed using the SRX software. The model conjugate was internalized by A2780 cancer cells via endocytosis, and most of the FITC signal (related to polymer) co-localized with lysosomes and late endosomes (FIG. 16). At 4 h, most of the FITC and Cy5 molecules were located at the margins of the cytoplasm and FITC-labeled HPMA copolymer molecules were surrounded by clusters of Cy5. This indicates that the conjugate was intact and localized in endosomes/lysosomes. At longer time intervals, an increasing amount of Cy5 molecules was found inside the cell, and the majority was located at a distance from the FITC-labeled HPMA copolymer. At 12 h, Cy5 molecules diffused all over the cell (FIG. 16). This shows that the side chains GFLG-Cy5 are cleaved by enzymes (cathepsin B) in the lysosomes, and the functional payload (i.e. Cy5 as drug model) is released and translocates into the cytoplasm.

### 2. Experimental Approach

i. SPECIFIC AIM 1. Design, Synthesis, and Characterization of Targeted, Long-Circulating HPMA Copolymer Conjugates with Ara-C and GDC-0980

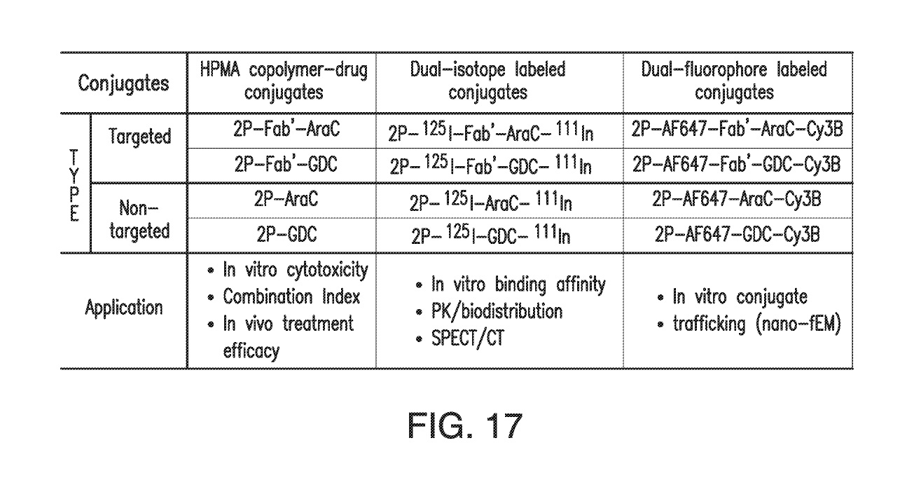

A new pathway for the synthesis of targeted long circulating backbone degradable polymeric drug delivery systems has been designed. In addition, the use of Fab′ fragments to produce targeted polymer conjugates allows better control over the size and composition of copolymers. The structure of the Fab′ introduces a unique SH group which functions as an attachment point for the polymer with maleimide side chains. Moreover, the smaller Fab′ fragment can provide a higher selectivity for tumor targeting than the entire antibody molecule or F(ab′)2. FIG. 17 is the list of main conjugates to be synthesized and evaluated.

a. Synthesis and characterization of backbone degradable, long-circulating, anti-CD33 Fab′-targeted HPMA copolymer-drug conjugates

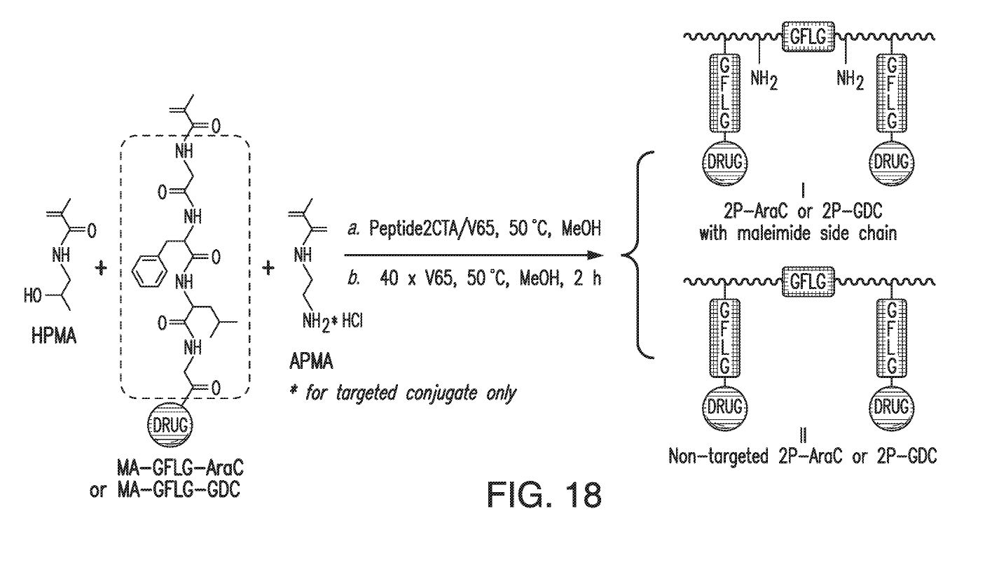

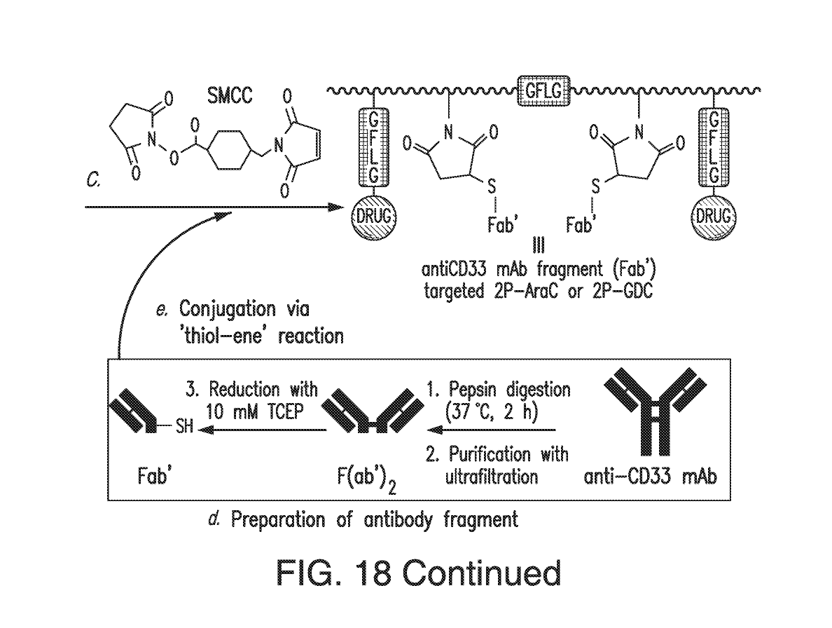

FIG. 18 summarizes the synthetic pathways and structure of the conjugates. The degradable diblock copolymers can be prepared by RAFT copolymerization of HPMA, N-methacryloylglycylphenylalanyl-leucylglycyl-drug (MA-GFLG-Drug), and N-(3-aminopropyl)methacrylamide (APMA) using peptide2CTA as chain transfer agent, followed by reaction with heterobifunctional linker SMCC to convert amino groups on side chains to maleimido groups. Anti-CD33 antibodies can be digested with 10% w/w pepsin, F(ab′)2 isolated and then reduced to Fab′ with 10 mM tris(2-carboxyethyl)phosphine and used for binding to maleimido groups on the polymer via thioether bonds. As control, non-targeted polymer-drug conjugates can be synthesized using a similar procedure but without APMA. This synthetic approach permits to vary the structure of the conjugates easily in order to evaluate in detail the relationship between structure and biological properties.

b. Synthesis and Characterization of Dual-Fluorophore Labeled Backbone Degradable, Long-Circulating, Anti-CD33 Fab′-Targeted HPMA Copolymer-Drug Conjugates

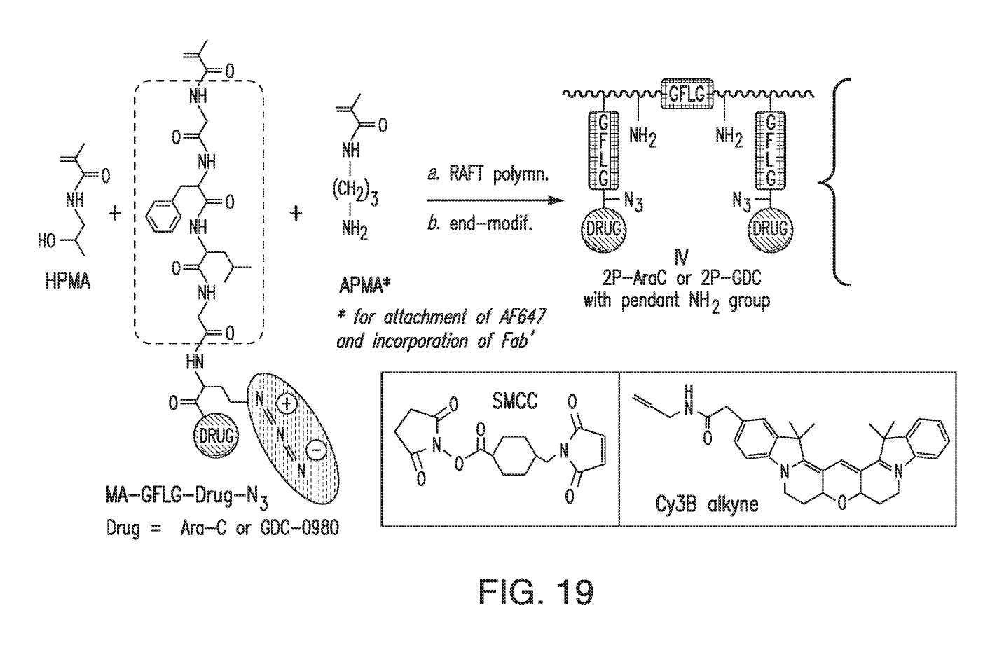

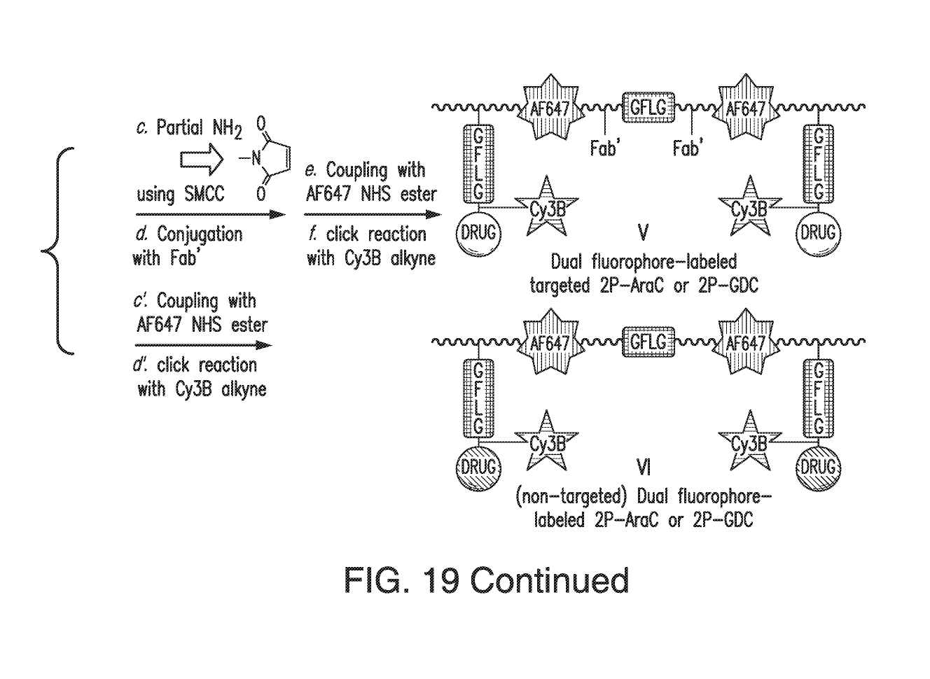

To synthesize dual-fluorophore labeled polymer conjugates, APMA can be used as comonomer to introduce pendant amino group for backbone labeling. For drug fate monitoring, the cleavable GFLG spacer can be extended by azidohomoalanine (FIG. 19). This permits attachment of imaging probes. In the lysosomes, cathepsin B can cleave the bond between glycine and azidohomoalanine, releasing a stable labeled drug.

In the next step, a fraction of amino groups at side chain termini can be converted into maleimido groups by reaction with SMCC (succinimidyl-4-(N-maleimidomethyl)cyclohexane-1-carboxylate), a heterobifunctional reagent. This can result in a polymer precursor that possesses three functional groups: the maleimido group to attach Fab′ antibody fragments, the amino groups to attach Alexa Fluor 647 via aminolysis of N-hydroxysuccinimide (NHS) ester groups, and the azido group for attachment of Cy3B via click reaction.

Non-targeted dual-labeled conjugates can be synthesized similarly but without attachment of Fab′ (FIG. 19).

c. Synthesis and Characterization of Dual-Radioisotope Labeled Backbone Degradable, Long-Circulating, Anti-CD33 Fab′-Targeted HPMA Copolymer-Drug Conjugates

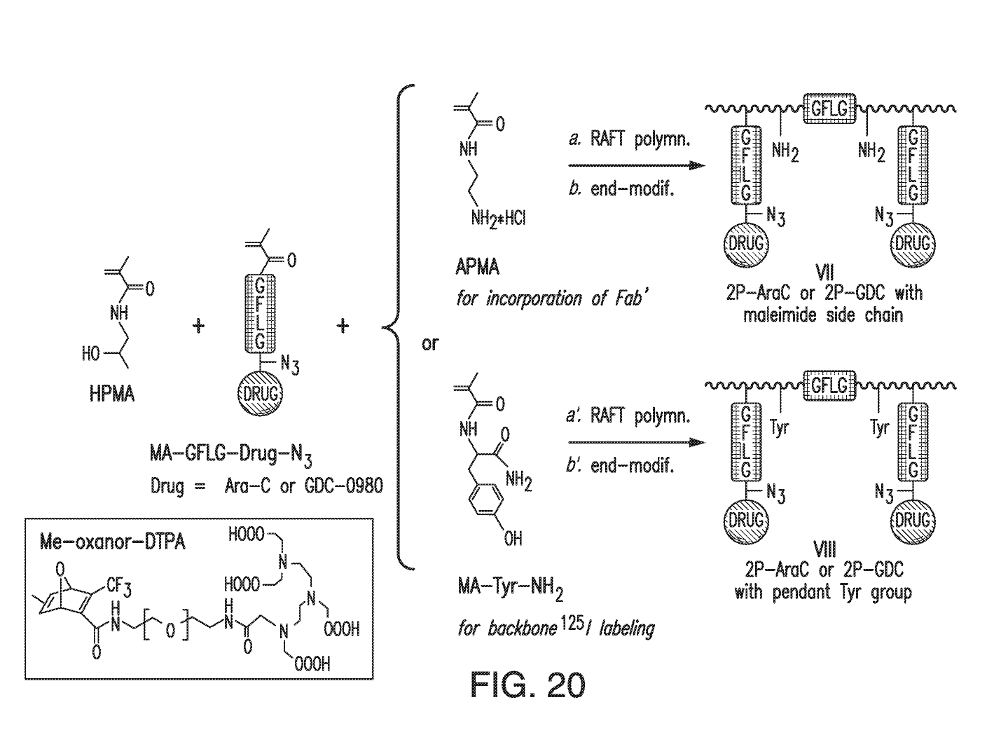

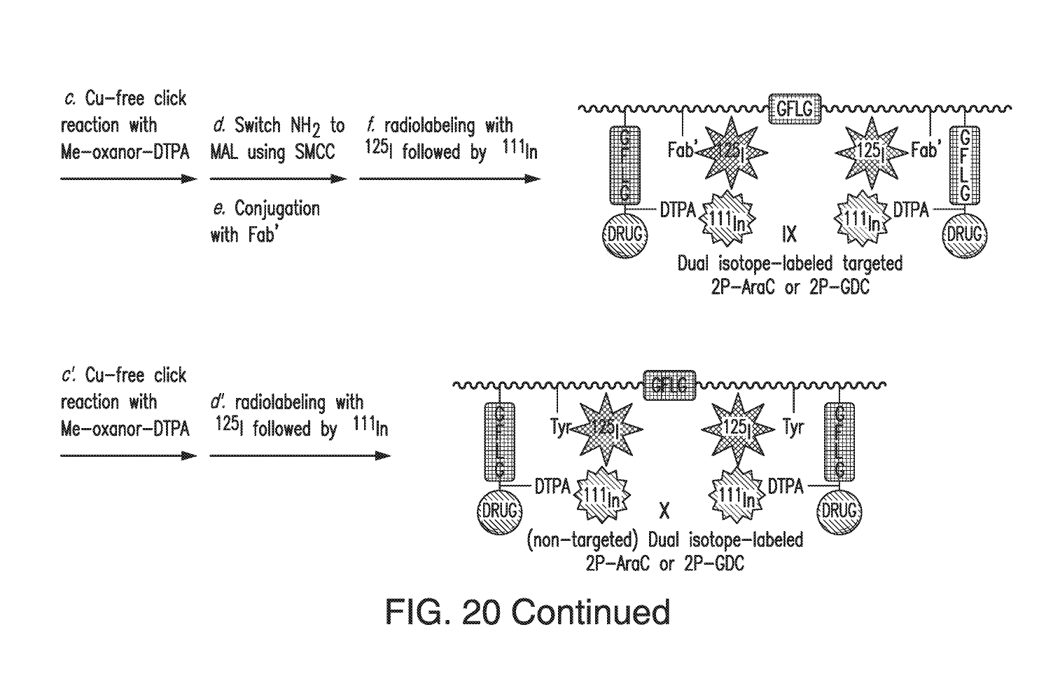

As described above, the polymerizable derivatives of drugs containing azido groups can be used for RAFT copolymerization. Me-oxanor-DTPA can be incorporated into GFLG spacer by click reaction for complexation of ¹¹¹In (FIG. 20). For Fab′ fragment binding, APMA can be added as comonomer into the polymerization mixture; Fab′ can be attached via thioether bonds by reaction with maleimido group at copolymer side chain termini. To monitor the fate of targeted conjugates, Fab′ can be radioiodinated with ¹²⁵I.

To synthesize non-targeted dual-isotope labeled conjugates, MA-Tyr-NH2 can replace APMA in copolymerization. The pendant -Tyr moieties can be used for ¹²⁵I labeling.

Physicochemical characterization of polymer precursors and conjugates can be performed by size exclusion chromatography, HPLC, amino acid analysis, and scintigraphy. The stability at different pH and enzyme-related degradation can be evaluated using RP-HPLC and UV-vis spectrophotometry.

ii. SPECIFIC AIM 2. In Vitro Evaluation of Macromolecular Combination Therapeutic Systems

The therapeutic potential of a targeting drug delivery system depends primarily on its targeting effect and internalization efficiency in targeted cells. The polymer-drug conjugates synthesized in Specific Aim 1 can be evaluated in vitro for binding affinity, internalization efficiency and cytotoxicity using various AML cell lines. Advanced imaging techniques such as nano-fEM can be used to investigate subcellular trafficking of these conjugates and drug release for structural optimization.

a. Determination of Binding Affinity and Internalization Efficiency of the Conjugates

a) Binding affinity: To prove that targeting specificity of Fab′-targeted drug delivery is related to CD33 expression, CD33+ leukemia cells HL-60 and CD33− cells Namalwa can be used. Cells can be incubated with ¹²⁵I-labeled conjugates. Both saturation/displacement binding studies can be conducted to assess nonspecific binding and targeting avidity of conjugates to leukemia cells. Briefly, the cells can be incubated at 4° C. with increasing amounts of ¹²⁵I-labeled conjugates in the presence of blocking compounds and counted for radioactivity after incubation. Non-targeted conjugates and free Fab′ can serve as controls. The data can be analyzed to obtain binding affinity of varying conjugates.

b) Internalization efficiency: Internalization assays can be performed to look at the ability of our Fab′-mediated drug delivery system to penetrate the cells. Cells can be incubated with ¹²⁵I-labeled conjugates. Radioactivity can be measured by gamma-spectrometry. Surface-bound conjugates can be stripped from the cell membrane using low pH (˜2.9) buffers, whereas internalized conjugates can remain unaffected. Consequently, the count of unstripped cells is assumed to represent total cell-associated conjugates (surface-bound and internalized), and the count of stripped cells represents the conjugates that have been internalized.

b. Monitoring of Subcellular Trafficking of Conjugates Using Nano-fEM (PALM/SEM)

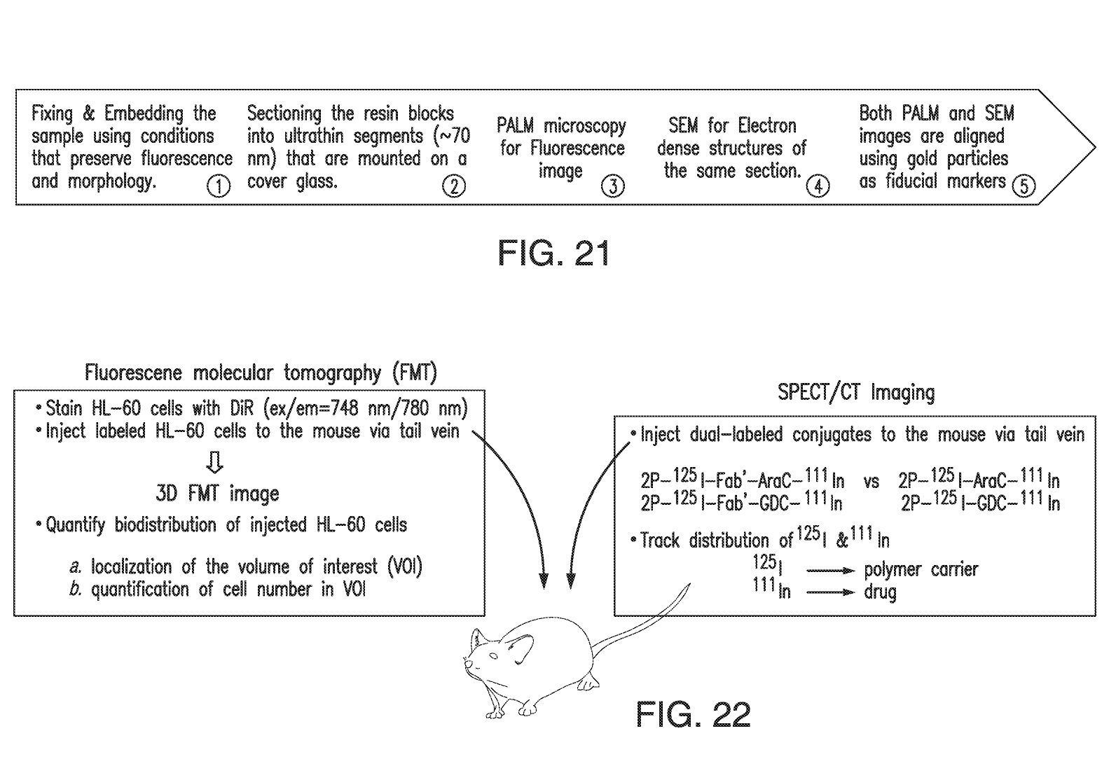

As described above, leukemia cells can be incubated with dual-fluorophore-labeled conjugates (2P-Fab′-AraC & 2P-Fab′-GDC) and observed using nano-resolution fluorescence electron microscopy (nano-fEM) that can map the distribution of the drug and polymeric carrier at nano-scale levels by imaging the same cell sections using PALM and SEM. The procedure shown in FIG. 21 can be followed. PALM can provide localization of Cy3B-labeled drug (ex/em=558/572 nm) and AF647-labeled Fab′-polymer carrier (ex/em=652/668nm) and subsequently their localization can be correlated with ultrastructural features revealed by SEM. Information on the drug trafficking path following internalization (from endocytosis to subcellular distribution) can be obtained. Importantly, the nano-fEM images can be quantitatively analyzed to obtain drug release rates from the conjugates by calculating the ratio of fluorescence signal intensity inside and outside lysosomes. Quantitative analysis also allows study of drug distribution in specific organelles such as lysosomes, cytoplasm and nucleus. In addition, different phases of drug delivery can be correlated with cellular morphologic changes indicative of cell death. These experiments can show distribution-activity relationship of P-Fab′-drug conjugates.

c. Determination of Synergy of Conjugate Combinations Against Leukemia Cells Lines Including Primary Patient Leukemia Cells

The cytotoxicity of conjugates 2P-Fab′-AraC/2P-Fab′-GDC can be assessed using the MTS cell viability assay to determine their therapeutic potential. Free drugs and non-targeted conjugates, 2P-AraC/2P-GDC, can be used for comparison. To systemically evaluate cytotoxicity of the conjugates, AML cell lines with different phenotypes according to the French American British classification can be used. These are: KG-1a (M1), HL-60 and Kasumi-1 (M2), NB-4 (M3), OCI-AML-3 (M4), and THP-1 (M5). All these lines express CD33. KG-1a cells express low CD33 levels and can be used as a negative control along with the lymphoma cell line Namalwa, which does not express CD33. The differences in cytotoxicity can be correlated with profiles of binding affinity, internalization efficiency and drug release rate, which can facilitate optimization of the drug delivery system.

To characterize the synergy of drug combinations, the factors such as incubation time, the dose ratio and the administration schedule can be examined. Synergism, additivity or antagonism can be determined using the combination index (CI) method of Chou and Talalay. Different incubation strategies can be used to identify the optimal combination regime with maximal augmentation of cytotoxicity. The effect of the treatments on leukemia cell proliferation ([3H]-Thymidine assay), apoptosis (Annexin V assay), clonogenicity (colony assay), differentiation, and cell cycle can be determined using well established techniques. In order to systemically evaluate toxicity of those treatments prior to in vivo study, normal human hematopoietic stem/progenitor cells exposed to the treatment can also be assessed in aforementioned assays.

iii. SPECIFIC AIM 3. In Vivo Evaluation of Targeted AML Therapy

Studies have shown significant synergy when using combination of Ara-C (cytarabine) and a PIO3k/mTOR inhibitor GDC-0980 compared with the current standard of cytarabine and daunorubicin (FIG. 8/FIG. 14). A large enhancement of anti-tumor activity when using long circulating conjugates compared to free drugs/low molecular weight conjugates was demonstrated (FIG. 8). Moreover, superior tumor growth inhibition in prostate cancer xenografts has been observed when targeting not only differentiated proliferative cancer cells but also self-renewal pathways (FIG. 11).

This study addresses the following questions: 1) Do the targeting delivery systems deliver the drugs to disease sites and leukemia cells in the required concentrations? 2) Do the Fab′-polymer-drug conjugates have the desired effect? 3) Which treatment protocol is the most effective one in animal tumor models?

a. Animal Model

NOD/SCID IL2Rγ null can be used because of their severely immunocompromised state and that readily allows engraftment of leukemia cells. Systemic leukemia that mimics the human disease can be performed as known in the art. Briefly, mice can receive a sublethal dose of radiation (250 cGy) in a single fraction. After a 24 hour rest period, mice can be inoculated intravenously through the tail vein with ˜2 to 5×10⁶ HL-60 or unsorted primary patient AML cells. In the latter case, engraftment can be obtained with almost 70% of patient isolates with 40% high-level engraftment (>10% AML cells in the bone marrow).

b. Dual-Isotope SPECT/CT Imaging Combined with FMT for Biodistribution and Pharmacokinetics Studies

To evaluate targeting of the conjugates to leukemia cells in vivo, FMT imaging can be used to localize contrast-tagged HL-60 leukemia cells after systemic inoculation of mice. Then dual-isotope SPECT/CT imaging can be applied to simultaneously track the fate of ¹²⁵I-labeled polymer and ¹¹¹In-labeled drugs in the same mouse after i.v. administration of dual-isotope labeled conjugates (see Table 17 and FIG. 22)

The FMT and SPECT/CT data can be correlated to assess the in vivo targeting efficiency of different polymer-drug conjugates (FIG. 22).

To confirm the findings in whole-body imaging, ex vivo measurements can also be performed. At different time intervals, 5 mice in each group can be sacrificed and tissues harvested. The biodistribution of DiR-labeled HL-60 cells in tissues can be obtained using FMT, while the biodistribution of drugs can be measured by gamma-spectrometry using ¹¹¹In protocol. One month later (after sufficient ¹¹¹In decay), the same samples can be measured again using gamma-spectrometry by ¹²⁵I protocol to determine biodistribution of polymeric carrier. In addition, blood samples of mice can be collected at predetermined time points and their radioactivity can be measured using ¹¹¹In/¹²⁵I protocols to obtain the pharmacokinetics profile of the polymeric carrier and drug. Urine can be collected in metabolic cages. The radioactivity of macromolecular carrier and drug excreted in the urine can be measured to determine the route of excretion. The integrated in vivo and ex vivo studies can provide quantitative information about biodistribution and pharmacokinetics, which can facilitate further optimization of the drug delivery systems.

c. Determination of the MTD and Therapeutic Efficacy of the Individual Conjugates in Mice

Gaussia luciferase (Gluc)-based bioluminescence imaging has been used to detect a single leukemia cell within 10,000 bone marrow cells in mice due to the extremely high sensitivity of Gluc (>1000-fold more sensitive than firefly luciferase and renilla luciferase). This demonstrates its potential for diagnosis of MRD in mice bearing leukemia cells. Thus, Gluc-based bioluminescence imaging can be used to determine the therapeutic efficacy of the conjugates in this study. To this end, HL-60 leukemia cells can be transduced to coexpress Gluc and eGFP. After intravenous administration of Gluc-expressing HL-60 cells to sublethally irradiated NOD/SCID IL2Rγ null mice, bioluminescence imaging can be performed to monitor leukemia cell engraftment. In separate experiments, drug treatments can start 1, 2 or 3 weeks after leukemia inoculation in order to correlate the therapeutic effect with different levels of leukemia burden. Five different doses of each conjugate can be injected intravenously to leukemia-bearing mice. Free drugs and non-targeted conjugates can be used as controls. Leukemia burden and body weight can be monitored. At different time intervals, 4 to 5 mice in each group can be sacrificed and leukemia engraftment can be evaluated by flow cytometry and immunohistochemistry and correlated with results from bioluminescence imaging. These experiments can also allow us to determine the MTD of the conjugates individually. When body weight loss exceeds 10% or mice become moribund (lethargy, ruffled skin, lack of motion, poor oral intake, diarrhea, and/or hind limb paralysis), mice can be sacrificed and histopathological examination of tissues can be performed. Based on the therapeutic efficacy and MTD, the optimal dose of each conjugate individually will be optimized.

d. Determination of the Therapeutic Efficacy of Conjugate Combinations

As described above, Gluc-expressing HL-60 cells will be i.v. injected to the sublethally irradiated NOD/SCID IL2Rγ null mice to establish the leukemia model; bioluminescence imaging can be performed to monitor leukemia cell engraftment and to determine the therapeutic efficacy. The combination of the 2 conjugates can be used to treat mice bearing Gluc-expressing HL-60 tumor. Treatment strategies including dose, dose ratio and administration schedule can be selected based on in vitro combination studies and in vivo pharmacokinetics as well as individual conjugate efficacy. The effect of the sequence of administration can be evaluated as follows: a) conjugate 1 followed by conjugate 2; b) conjugate 2 followed by conjugate 1; and c) simultaneous administration of both conjugates. Leukemia engraftment will be monitored by bioluminescence imaging as described above. Mice can be monitored for evidence of treatment-related toxicity. Single-conjugate treatment and free-drug combinations can also be tested and compared with the conjugate combination treatment.

e. Evaluation of the Therapeutic Efficacy of Conjugate Combinations on Patient-Derived AML Model.

Having identified the optimal conjugate combination protocol in the work described above, the effect of these combinations can be studied on AML patient cell isolates to extend the observations to other leukemia cell lines. Briefly, patient-derived AML cells can be transduced with a lentivirus encoding Gluc and eGFP before transplantation in mice. Mice can receive combination treatments and can be imaged three times weekly using bioluminescence imaging to determine tumor response. In order to determine the effect of leukemia load on response to therapy, different groups of mice can start receiving treatment starting 1, 2, and 3 weeks after inoculation with leukemia cells. Mice can be sacrificed at weekly intervals and imaging data can be correlated with data on leukemic cell engraftment in hematopoietic organs (bone marrow, spleen, and liver) using immunohistochemistry and flow cytometry.

f. Use of Multiplexed Monoclonal Antibodies (mAb) to Evaluate Therapeutic Efficacy and Detect MRD

The experiments proposed above rely on ex vivo modification of leukemic cells to allow detection by imaging. This is a powerful approach for development and optimization purposes, but it does not reproduce actual clinical conditions. We will therefore use drug combinations in experiments with in vivo imaging to study AML patient isolates that have not been manipulated ex vivo. Xenografts can be set up as above and treatments started 1, 2, or 3 weeks after leukemia cell inoculation. Response to therapy can be evaluated on a weekly basis using fluorescence molecular tomography imaging after injecting animals with a cocktail of 3 multiplexed mAbs conjugated with Alexa Fluor 680. The mAbs can include an anti-CD33 antibody and 2 others chosen based on the phenotype of the individual leukemic cells used. Mice can be sacrificed at regular intervals and leukemia engraftment can be evaluated by flow cytometry and immunohistochemistry to correlate with imaging data. This strategy can also lay the foundation for future studies to detect MRD by modified [18F]fluorothymidine PET imaging while distinguishing normal from malignant CD33+ cells through detection of the unique aberrant phenotype of leukemic cells. Finally, in separate experiments, serial transplantation experiments of AML cells in NOD/SCID IL2Rγ null mice can be used in order to determine the effect of the drugs on leukemia initiating cells and therefore LSCs.

Data analysis. The Student's t test (assuming unpaired variables and unequal variance between samples) can be used to test differences in therapeutic efficiency, organ uptakes, pharmacokinetic parameters, and toxicity among different conjugates. Comparison among groups can be performed using one-way ANOVA. The significance level can be set at 0.05. If distributions are excessively skewed the rank-based Wilcoxon test can be used. Sample size projection was determined using normal power calculations. With 6 mice per group, there would be a 90% power to detect a difference of 2 standard deviations between groups. Experiments with 10 mice per group can be set up in order to take into account engraftment failure or loss of animals during manipulation for causes unrelated to the treatments.

Those skilled in the art will recognize, or be able to ascertain using no more than routine experimentation, many equivalents to the specific embodiments of the method and compositions described herein. Such equivalents are intended to be encompassed by the following claims.

### REFERENCES

[1] R. Siegel, J. Ma, Z. Zou, A. Jemal, C A. Cancer J. Clin. 2014, 64, 9.
[2] J. W. Yates, H. J. Wallace, Jr., R. R. Ellison, J. F. Holland, Cancer Chemother. Rep. 1973, 57, 485.
[3] A. K. Burnett, R. K. Hills, D. W. Milligan, A. H. Goldstone, A. G. Prentice, M. F. McMullin, A. Duncombe, B. Gibson, K. Wheatley, J. Clin. Oncol. 2010, 28, 586.
[4] T. Buchner, W. E. Berdel, C. Haferlach, T. Haferlach, S. Schnittger, C. Muller-Tidow, J. Braess, K. Spiekermann, J. Kienast, P. Staib, A. Gruneisen, W. Kern, A. Reichle, G. Maschmeyer, C. Aul, E. Lengfelder, M. C. Sauerland, A. Heinecke, B. Wormann, W. Hiddemann, J. Clin. Oncol. 2009, 27, 61.
[5] G. J. Roboz, Curr. Opin. Oncol. 2012, 24, 711.
[6] H. F. Fernandez, Z. Sun, X. Yao, M. R. Litzow, S. M. Luger, E. M. Paietta, J. Racevskis, G. W. Dewald, R. P. Ketterling, J. M. Bennett, J. M. Rowe, H. M. Lazarus, M. S. Tallman, N Engl. J. Med. 2009, 361, 1249.
[7] F. Mandelli, M. Vignetti, S. Suciu, R. Stasi, M. C. Petti, G. Meloni, P. Muus, F. Marmont, J. P. Marie, B. Labar, X. Thomas, F. Di Raimondo, R. Willemze, V. Liso, F. Ferrara, L. Baila, P. Fazi, R. Zittoun, S. Amadori, T. de Witte, J. Clin. Oncol. 2009, 27, 5397.
[8] S. A. Johnson, Expert Opin. Pharmacother. 2001, 2, 929.
[9] E. L. Schenk, B. D. Koh, K. S. Flatten, K. L. Peterson, D. Parry, A. D. Hess, B. D. Smith, J. E. Karp, L. M. Karnitz, and S. H. Kaufmann, Clin. Cancer Res. 2012, 18, 5364.
[10] D. P. Sutherlin, L. Bao, M. Berry, G. Castanedo, I. Chuckowree, J. Dotson, A. Folks, L. Friedman, R. Goldsmith, J. Gunzner, T. Heffron, J. Lesnick, C. Lewis, S. Mathieu, J. Murray, J. Nonomiya, J. Pang, N. Pegg, W. W. Prior, L. Rouge, L. Salphati, D. Sampath, Q. Tian, V. Tsui, N. C. Wan, S. Wang, B. Wei, C. Wiesmann, P. Wu, B. Y. Zhu, A. Olivero, J. Med. Chem. 2011, 54, 7579.
[11] J. J. Wallin, K. A. Edgar, J. Guan, M. Berry, W. W. Prior, L. Lee, J. D. Lesnick, C. Lewis, J. Nonomiya, J. Pang, L. Salphati, A. G. Olivero, D. P. Sutherlin, C. O'Brien, J. M. Spoerke, S. Patel, L. Lensun, R. Kassees, L. Ross, M. R. Lackner, D. Sampath, M. Belvin, L. S. Friedman, Mol. Cancer Ther. 2011, 10, 2426.
[12] L. Salphati, J. Pang, E. G. Plise, L. B. Lee, A. G. Olivero, W. W. Prior, D. Sampath, S. Wong, X. Zhang, Drug Metab. Dispos. 2012, 40, 1785.
[13] D. P. English, S. Bellone, E. Cocco, I. Bortolomai, S. Pecorelli, S. Lopez, D. A. Silasi, P. E. Schwartz, T. Rutherford, A. D. Santin, Am. J. Obstet. Gynecol. 2013, 209, 465 el.
[14] P. J. Shami, J. E. Saavedra, L. Y. Wang, C. L. Bonifant, B. A. Diwan, S. V. Singh, Y. Gu, S. D. Fox, G. S. Buzard, M. L. Citro, D. J. Waterhouse, K. M. Davies, X. Ji, L. K. Keefer, Mol. Cancer Ther. 2003, 2, 409.
[15] A. E. Maciag, H. Chakrapani, J. E. Saavedra, N. L. Morris, R. J. Holland, K. M. Kosak, P. J. Shami, L. M. Anderson, L. K. Keefer, J. Pharmacol. Exp. Ther. 2011, 336, 313.
[16] J. Kitagaki, Y. Yang, J. E. Saavedra, N. H. Colburn, L. K. Keefer, A. O. Perantoni, Oncogene 2009, 28, 619.
[17] A. Weyerbrock, N. Osterberg, N. Psarras, B. Baumer, E. Kogias, A. Werres, S. Bette, J. E. Saavedra, L. K. Keefer, A. Papazoglou, Neurosurgery 2012, 70, 497.
[18] P. J. Shami, J. E. Saavedra, C. L. Bonifant, J. Chu, V. Udupi, S. Malaviya, B. I. Carr, S. Kar, M. Wang, L. Jia, X. Ji, L. K. Keefer, J. Med. Chem. 2006, 49, 4356.
[19] D. M. Townsend, K. D. Tew, Oncogene 2003, 22, 7369.
[20] G. Zhao, X. Wang, Curr. Med. Chem. 2006, 13, 1461.
[21] Y. C. Awasthi, R. Sharma, S. S. Singhal, Int. J. Biochem. 1994, 26, 295.
[22] P. Baines, P. Cumber, R. A. Padua, Baillieres Clin. Haematol. 1992, 5, 943.
[23] J. R. Matthews, C. H. Bating, M. Panico, H. R. Morris, R. T. Hay, Nucleic Acids Res. 1996, 24, 2236.
[24] N. Nath, M. Chattopadhyay, L. Pospishil, L. Z. Cieciura, S. Goswami, R. Kodela, J. E. Saavedra, L. K. Keefer, K. Kashfi, Biochem. Pharmacol. 2010, 80, 1641.
[25] J. E. Saavedra, A. Srinivasan, C. L. Bonifant, J. Chu, A. P. Shanklin, J. L. Flippen-Anderson, W. G. Rice, J. A. Turpin, K. M. Davies, L. K. Keefer, J. Org. Chem. 2001, 66, 3090.
[26] Y. Mitsukami, M. S. Donovan, A. B. Lowe, C. L. McCormick, Macromolecules 2001, 34, 2248.
[27] J. Kopeček, H. Bažilová, Eur. Polym. J. 1973, 9, 7.
[28] J. Kopeček, P. Rejmanová, J. Strohalm, B. RŘíhová, V. Chytrý, J. B. Lloyd, R. Duncan, U.S. Pat. No. 5,037,883, 1991.
[29] Šubr V, Ulbrich K, React. Funct. Polym. 2006, 66, 1525.
[30] J. Yang, K. Luo, H. Pan, P. Kopečková, J. Kopeček, React. Funct. Polym. 2011, 71, 294.
[31] Y. Zhou, J. Yang, R. Zhang, J. Kopeček, Eur. J. Pharm. Biopharm. 2014, 89, 107.
[32] T. C. Chou, R. J. Motzer, Y. Tong, G. J. Bosl, J. Natl. Cancer Inst. 1994, 86, 1517.
[33] R. Zhang, K. Luo, J. Yang, M. Sima, Y. Sun, M. M. Janát-Amsbury, J. Kopeček, J. Control. Release 2013, 166, 66.
[34] R. Zhang, J. Yang, M. Sima, Y. Zhou, J. Kopeček, Proc. Natl. Acad. Sci. U.S.A. 2014, 111,12181.
[35] S. Park, N. Chapuis, J. Tamburini, V. Bardet, P. Comillet-Lefebvre, L. Willems, A. Green, P. Mayeux, C. Lacombe, D. Bouscary, Haematologica 2010, 95, 819.
[36] G. J. Roboz, M. Guzman, Expert Rev. Hematol. 2009, 2, 663.
[37] B. Moshaver, A. van Rhenen, A. Kelder, M. van der Pol, M. Terwijn, C. Bachas, A. H. Westra, G. J. Ossenkoppele, S. Zweegman, G. J. Schuurhuis, Stem Cells 2008, 26, 3059.
[38] A. J. Gentles, S. K. Plevritis, R. Majeti, A. A. Alizadeh, JAMA 2010, 304, 2706.
[39] M. L. Guzman, S. J. Neering, D. Upchurch, B. Grimes, D. S. Howard, D. A. Rizzieri, S. M. Luger, C. T. Jordan, Blood 2001, 98, 2301.
[40] S. Park, N. Chapuis, V. Bardet, J. Tamburini, N. Gallay, L. Willems, Z. A. Knight, K. M. Shokat, N. Azar, F. Viguie, N. Ifrah, F. Dreyfus, P. Mayeux, C. Lacombe, D. Bouscary, Leukemia 2008, 22, 1698.
[41] J. Zhou, J. Wulfkuhle, H. Zhang, P. Gu, Y. Yang, J. Deng, J. B. Margolick, L. A. Liotta, E. Petricoin, Y. Zhang, Proc. Natl. Acad. Sci. U.S.A. 2007, 104, 16158.
[42] N. Sandhofer, K. H. Metzeler, M. Rothenberg, T. Herold, S. Tiedt, V. Groiss, M. Carlet, G. Walter, T. Hinrichsen, O. Wachter, M. Grunert, S. Schneider, M. Subklewe, A. Dufour, S. Frohling, H. G. Klein, W. Hiddemann, I. Jeremias, K. Spiekermann, Leukemia 2015, 29, 828.
[43] A. Dubrovska, S. Kim, R. J. Salamone, J. R. Walker, S. M. Maira, C. Garcia-Echeverria, P. G. Schultz, V. A. Reddy, Proc. Natl. Acad. Sci. U.S.A. 2009, 106, 268.
[44] G. J. Roboz, T. Rosenblat, M. Arellano, M. Gobbi, J. K. Altman, P. Montesinos, C. O'Connell, S. R. Solomon, A. Pigneux, N. Vey, R. Hills, T. F. Jacobsen, A. Gianella-Borradori, O. Foss, S. Vetrhusand, F. J. Giles, J. Clin. Oncol. 2014, 32, 1919.
[45] E. J. Feldman, J. E. Lancet, J. E. Kolitz, E. K. Ritchie, G. J. Roboz, A. F. List, S. L. Allen, E. Asatiani, L. D. Mayer, C. Swenson, A. C. Louie, J. Clin. Oncol. 2011, 29, 979.
[46] J. Kopeček, Adv. Drug Deliv. Rev. 2013, 65, 49.
[47] J. Yang, J. Kopeček, J. Control. Release 2014, 190, 288.
[48] J. Kopeček, P. Kopečkova, Adv. Drug Deliv. Rev. 2010, 62, 122.
[49] T. Minko, P. Kopečkova, V. Pozharov, J. Kopeček, J. Control. Release 1998, 54, 223.
[50] C. L. Willman, Leukemia 1996, 10 Suppl 1, S33.
[51] A. F. List, Oncology (Williston Park) 1993, 7, 23.
[52] R. J. Arceci, Blood 1993, 81, 2215.
[53] W. L. Xu, H. L. Shen, Z. F. Ao, B. A. Chen, W. Xia, F. Gao, Y. N. Zhang, Leuk. Res. 2006, 30, 407.

We claim:

1. A method of treating AML comprising administering an effective amount of a first AML therapeutic and an effective amount of a second AML therapeutic,
wherein the first AML therapeutic is cytarabine, daunorubicin, GDC-0980, arylated diazeniumdiolate, or derivatives thereof,
wherein the second AML therapeutic is cytarabine, daunorubicin, GDC-0980, arylated diazeniumdiolate, or derivatives thereof, and
wherein the first and second AML therapeutics are not cytarabine and anthracycline.

2. The method of claim 1, wherein the second AML therapeutic is different from the first AML therapeutic.

3. The method of claims 1-2, wherein at least one of the AML therapeutics is cytarabine.

4. The method of claims 1-3, wherein at least one of the AML therapeutics is GDC-0980.

5. The method of claims 1-4, wherein the first and second AML therapeutics provide a synergistic effect.

6. The method of claims 1-5, wherein the first and second AML therapeutics are formulated in a single composition.

7. The method of claims 1-6, wherein the first and second AML therapeutics are formulated in separate compositions.

8. The method of claims 7, wherein the first and second AML therapeutics are administered simultaneously.

9. The method of claim 8, wherein the first and second AML therapeutics are administered consecutively.

10. The method of claims 1-9, wherein the first AML therapeutic is cytarabine and the second AML therapeutic is GDC-0980.

11. A method of treating AML comprising administering an effective amount of a first AML therapeutic conjugated to N-(2-hydroxypropyl)methacrylamide (HPMA) copolymer.

12. The method of claim 0, further comprising a second AML therapeutic.

13. The method of claim 0, wherein the second AML therapeutic is conjugated to HPMA copolymer.

14. The method of claim 0-0, wherein the first and second AML therapeutics provide a synergistic effect.

15. The method of claim 0-0, wherein the first AML therapeutic conjugated to HPMA copolymer comprises cytarabine, daunorubicin, GDC-0980, arylated diazeniumdiolate, or derivatives thereof.

16. The method of claims 0-0, wherein the second AML therapeutic conjugated to HPMA copolymer comprises cytarabine, daunorubicin, GDC-0980, arylated diazeniumdiolate, or derivatives thereof.

17. The method of claim 0, wherein the second AML therapeutic conjugated to HPMA copolymer is different than the first AML therapeutic conjugated to HPMA copolymer.

18. The method of claims 0-0, wherein at least one of the AML therapeutics conjugated to HPMA copolymer is cytarabine.

19. The method of claims 0-0, wherein the first and second AML therapeutics conjugated to HPMA copolymer are formulated in a single composition.

20. The method of claims 0-0, wherein the first and second AML therapeutics conjugated to HPMA copolymer are formulated in separate compositions.

21. The method of claim 0, wherein the first and second AML therapeutics conjugated to HPMA copolymer are administered simultaneously.

22. The method of claim 0, wherein the first and second AML therapeutics conjugated to HPMA copolymer are administered consecutively.

23. The method of claims 0-0, wherein at least one of the AML therapeutics conjugated to HPMA copolymer comprise a GFLG linker.

24. The method of claims 0-0, wherein at least one of the AML therapeutics conjugated to HPMA copolymer further comprises a targeting moiety.

25. The method of claim 0, wherein the targeting moiety is an antibody or fragment thereof.

26. The method of claims 0-0, wherein the first AML therapeutic conjugated to HPMA copolymer comprises a HPMA copolymer backbone comprising at least two HPMA copolymer segments connected by a degradable peptide sequence.

27. The method of claim 0, wherein the second AML therapeutic conjugated to HPMA copolymer comprises a HPMA copolymer backbone comprising at least two HPMA copolymer segments connected by degradable peptide sequences.

28. The method of claims 0-0, wherein the first AML therapeutic conjugated to HPMA copolymer comprises at least two AML therapeutics conjugated to the HPMA copolymer backbone.

29. The method of claims 0-0, wherein the second AML therapeutic conjugated to HPMA copolymer comprises at least two AML therapeutics conjugated to the HPMA copolymer backbone.

30. The method of claims 0-0, wherein the AML therapeutics conjugated to HPMA copolymer comprise a GFLG linker.

31. The method of claims 0-0, wherein the HPMA copolymer backbone comprises a GFLG linker.

32. The method of claims 0-0, wherein the HPMA copolymer backbone further comprises a targeting moiety.

33. The method of claim 0, wherein the targeting moiety comprises an antibody or fragment thereof.

34. The method of claim 0, wherein the antibody or fragment thereof comprises an Fab region.

35. The method of claims 0-0, wherein the antibody or fragment thereof is an anti-CD33 antibody or fragment thereof.

36. The method of claims 11-35, wherein the first AML therapeutic is cytarabine.

37. The method of claim 12, wherein the second AML therapeutic is GDC-0980.

38. The method of claim 37, wherein the GDC-0980 is conjugated to a HPMA copolymer.
